(19) United States
(12) Patent Application Publication
(10) Pub. No.: US 2025/0331901 A1
(43) Pub. Date: Oct. 30, 2025

(54) Bone Plating Assembly Having a Screw Guide, And Related Systems And Methods

(71) Applicant: DePuy Synthes Products, Inc., Raynham, MA (US)

(72) Inventors: John M. Noon, Swarthmore, PA (US); Emma Phillips, West Chester, PA (US); Michael Brace, Lansdale, PA (US)

(21) Appl. No.: 18/650,295

(22) Filed: Apr. 30, 2024

(51) Int. Cl. A61B 17/80

(52) U.S. Cl. CPC A61B 17/808; A61B 17/8076

(57) ABSTRACT

A screw guide for connecting to a bone plate includes a body having proximal and distal ends spaced from each other along a direction. The distal end has a coupling mechanism for coupling to the bone plate at an orientation. The body defines at least one channel that extends along a central axis and is configured to receive a bone fastener therein in a pre-loaded position. The coupling mechanism is configured such that the central axis substantially aligns with a central axis of a fastener hole of the bone plate when coupled to the bone plate at the orientation. The body has a retention mechanism at least partially located within the at least one channel and configured to apply a retention force to the bone fastener for retaining the bone fastener within the channel in the pre-loaded position.

### TECHNICAL FIELD

The present invention relates to bone fixation devices, and more particularly to bone plating devices for holding and guiding pre-loaded bone fasteners through respective holes of a bone plate.

### BACKGROUND

The process of bone fixation using a plate involves a number of steps. The process may include the steps of aligning and engaging a fastener, for example a screw, with the plate. Failure to properly align one of the fasteners with the plate may result in problems, such as generation of a burr. For example, improper alignment of a locking screw may result in generation of a burr as threads on a head of the locking screw head engage with threads of a hole of the plate, or when threads on a shaft of a screw rub against an edge of the hole of the plate.

Another potential consequence of improper alignment of a fastener with the hole of the plate is improper engagement of the head of the fastener, for example the threads on the head of the locking screw, and the hole of the plate, for example the threads within the hole of the plate. Improper engagement of the head of the fastener and the hole of the plate may lead to inadequate securement of the fastener to the plate, stripping of the corresponding threads of at least one of the plate and the screw, or both, any of which may contribute to malunion or non-union of a fractured bone to which the plate is attached.

The difficulty of aligning the fastener with a hole of the bone plate may be increased in certain situations such as when access to the plate is limited, for example during a minimally invasive surgery, and/or when using a self-drilling screw. A self-drilling screw may be inserted without a pilot hole or guide hole being pre-drilled into the target bone to be fixed. Thus using a self-drilling screw may eliminate some of the steps in a bone fixation process, for example the steps associated with pre-drilling a guide hole. However, at least some of the benefit of using a self-drilling screw may be offset by the increased difficulty from the alignment and the engagement steps of the process.

A device, kit, system, method, or any combination thereof that mitigates the difficulty of aligning and engaging a fastener with a hole of the bone plate may result in an increase in efficiency for bone fixation processes.

### SUMMARY

According to an embodiment of the present disclosure, a bone plating assembly for bone fixation includes a bone plate having a plate body that has an outer surface and a bone-facing surface opposite the outer surface and defining at least one plate hole that extends from the outer surface to the bone facing surface along a central hole axis. The bone plating assembly includes a guide body having a proximal end and a distal end spaced from each other along a direction. The distal end has a coupling mechanism for coupling to the bone plate at an orientation of the guide body. The guide body also defines at least one channel that extends along a central channel axis and is configured to receive at least one respective bone fastener therein in a pre-loaded position. The guide body is configured such that the central channel axis is substantially coaxial with the central hole axis when the guide body is connected to the bone plate at the orientation. The guide body includes a retention mechanism that is located within the at least one channel and is configured to apply a retention force to the at least one respective bone fastener for retaining the at least one respective bone fastener within the at least one channel in the pre-loaded position.

According to another embodiment of the present disclosure, a screw guide is configured for connecting to a bone plate and includes a body having proximal and distal ends spaced from each other along a direction. The distal end has a coupling mechanism for coupling to the bone plate at an orientation. The body defines at least one channel that extends along a central axis and is configured to receive a bone fastener therein in a pre-loaded position. The coupling mechanism is configured such that the central axis substantially aligns with a central axis of a screw hole of the bone plate when coupled to the bone plate at the orientation. The body has a retention mechanism at least partially located within the at least one channel and configured to apply a retention force to the bone fastener for retaining the bone fastener within the channel in the pre-loaded position.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a perspective view of a bone plate system that includes a pre-loaded plating assembly having a screw guide that carries pre-loaded bone screws and has a bone plate coupled thereto, according to an embodiment of the present disclosure;

FIG. 1B is an exploded, perspective view of the pre-loaded plating assembly illustrated in FIG. 1A, showing the screw guide, bone screws, and bone plate of the pre-loaded plating assembly;

FIG. 1C is a perspective view of the pre-loaded plating assembly illustrated in FIG. 1B;

FIG. 1D is a sectional side view of the pre-loaded plating assembly, taken along section line 1D-1D illustrated in FIG. 1C, and showing the bone screws retained in the screw guide in a pre-loaded position;

FIG. 1E is a perspective view of the screw guide illustrated in FIG. 1B, showing a screw retention mechanism of the screw guide, according to an embodiment of the present disclosure;

FIG. 1F is a sectional end view of a portion of the screw retention mechanism taken along section line 1F-1F illustrated in FIG. 1E;

FIG. 2A is a top view of the bone plate illustrated in FIG. 1B;

FIG. 2B is an enlarged view of a portion of the bone plate illustrated in FIG. 2A;

FIG. 2C is a bottom perspective view of the bone plate illustrated in FIG. 2A;

FIG. 2D is a top perspective view of the bone plate illustrated in FIG. 2A positioned atop bone;

FIG. 3A is a top perspective view of the screw guide and bone plate illustrated in FIG. 1B, shown in a coupling orientation but prior to coupling;

FIG. 3B is a bottom perspective view of the screw guide and bone plate in the coupling orientation illustrated in FIG. 3A, further showing a coupling mechanism of the screw guide;

FIG. 3C is a sectional side view of the screw guide and bone plate in the coupling orientation, taken along section line 3C-3C illustrated in FIG. 3A;

FIG. 3D is a perspective view of a portion of the coupling mechanism of the screw guide illustrated in FIG. 3B;

FIG. 3E is a side-by-side comparison of a bottom view of the screw guide and a top view of the bone plate illustrated in FIG. 3A;

FIG. 3F is a bottom view of the bone plate and the screw guide of FIG. 3A, shown coupled together;

FIG. 3G is a bottom perspective view of the bone plate and the screw guide of FIG. 3A, shown coupled together;

FIGS. 4A-4D are bottom views of bone plates according to additional embodiments, shown coupled to the screw guide illustrated in FIG. 1B; in particular, FIG. 4A shows an “X-plate”; FIG. 4B shows an “H-plate”; FIG. 4C shows a “double-T” or “pi” plate; and FIG. 4D shows a “ladder plate”;

FIG. 5A is a top perspective view of a “wide” or “large” version of the screw guide and bone plate illustrated in FIG. 1B, showing the screw guide and bone plate in a coupling orientation, according to an embodiment of the present disclosure;

FIG. 5B is a bottom perspective view of the screw guide and bone plate illustrated in FIG. 5A in the coupling orientation;

FIG. 5C is a bottom view of the bone plate and the screw guide of FIGS. 5A-5B, shown coupled together;

FIGS. 6A, 6B, and 6C are top views showing “wide” or “large” versions of the X-plate, H-plate, and ladder plate illustrated in FIGS. 4A, 4B, and 4D, respectively, according to embodiments of the present disclosure;

FIG. 7A is a top perspective view of an extended-area screw guide in a coupling orientation with the X-plate illustrated in FIG. 4A, according to an embodiment of the present disclosure;

FIG. 7B is a bottom perspective view of the extended-area screw guide and X-plate illustrated in FIG. 5A, shown in the coupling orientation;

FIG. 7C is a top view of the extended-area screw guide and X-plate of FIG. 7A, shown coupled together;

FIG. 7D is a bottom view of the extended-area screw guide and X-plate of FIG. 7A, shown coupled together;

FIG. 8A is a bottom perspective view of a “wide” or “large” version of the extended-area screw guide and X-plate illustrated in FIG. 7A, shown in a coupling orientation, according to an embodiment of the present disclosure;

FIG. 8B is a bottom view of the screw guide and X-plate of FIG. 8A, shown coupled together;

FIG. 9A is a top perspective view of an extended-area, multi-body screw guide in a coupling orientation with a “star plate”, according to an embodiment of the present disclosure;

FIG. 9B is a bottom perspective view of the screw guide and star plate illustrated in FIG. 9A, shown in the coupling orientation;

FIG. 9C is a side-by-side comparison of a top view of the star plate and a bottom view of the screw guide illustrated in FIG. 9A;

FIG. 9D is a bottom view of the screw guide and star plate of FIG. 9A, shown coupled together;

FIG. 9E is a bottom perspective exploded view of the screw guide illustrated in FIG. 9A, showing the multi-body nature of the screw guide;

FIG. 9F is a top perspective exploded view of the multi-body screw guide illustrated in FIG. 9A

FIGS. 10A-10B are perspective views of a single-channel screw guide, according to an embodiment of the present disclosure;

FIG. 10C is a bottom plan view showing the single-channel screw guide coupled to an exemplary “straight” bone plate;

FIG. 11 is a perspective view of a screw guide having another embodiment of a screw retention mechanism;

FIG. 12A is a perspective sectional view of a screw guide having an additional embodiment of a screw retention mechanism;

FIG. 12B is an enlarged sectional view of the screw retention mechanism illustrated in FIG. 12A;

FIG. 13A is a perspective sectional view of a screw guide having a further embodiment of a screw retention mechanism;

FIG. 13B is a bottom view of a portion of the screw guide showing the screw retention mechanism illustrated in FIG. 13A;

FIG. 14A is a top view of a bone plate for use with a screw guide, according to an embodiment of the present disclosure;

FIG. 14B is an enlarged view of a portion of the bone plate illustrated in FIG. 14A;

FIG. 14C is a bottom perspective view of the bone plate illustrated in FIG. 14A;

FIG. 14D is a top perspective view of the bone plate illustrated in FIG. 14A positioned atop bone;

FIG. 15A is a side-by-side comparison of a bottom view of a screw guide, according to an embodiment of the present disclosure, and the top view of the bone plate illustrated in FIG. 14A;

FIG. 15B is a bottom view of the bone plate and the screw guide illustrated in FIG. 15A, shown coupled together;

FIG. 15C is a side, partial section view of the bone plate and screw guide illustrated in FIG. 15A, shown in a coupling configuration;

FIGS. 16A-16C are bottom views of bone plates according to additional embodiments, shown coupled to the screw guide illustrated in FIG. 15A; in particular, FIG. 16A shows an X-plate; FIG. 16B shows an H-plate; and FIG. 16C shows a star plate;

FIGS. 17A and 17B are perspective views of a screw guide having spring arms having protrusions that couple with recesses of a bone plate, according to an embodiment of the present disclosure;

FIG. 17C is a partially exploded, perspective view of the screw guide illustrated in FIGS. 17A-17B;

FIGS. 18A and 18B are perspective views of a screw guide having protrusions that couple with exterior and interior recesses of a bone plate, according to an embodiment of the present disclosure;

FIG. 18C is a partially exploded, perspective view of the screw guide illustrated in FIGS. 18A-18B;

FIG. 18D is a bottom view of a portion of the bone plate illustrated in FIGS. 18A-18C, showing recesses in the bone plate;

FIG. 19A is a perspective view of a screw guide having a screw cartridge that is insertable within an outer housing, according to an embodiment of the present disclosure;

FIGS. 19B-19C are partially exploded, perspective views of the screw guide illustrated in FIG. 19A;

FIG. 20A is a perspective view of an additional-hole screw guide having a screw cartridge that is insertable within an outer housing, according to an embodiment of the present disclosure;

FIG. 20B is a perspective view of the outer housing of the screw guide illustrated in FIG. 20A;

FIG. 20C is an exploded perspective view of the screw guide illustrated in FIG. 20A;

FIG. 21A is a side view of a single-channel, dual-end screw guide, according to an embodiment of the present disclosure;

FIGS. 21B and 21C are respective perspective views of a first end (FIG. 21B) and a second end (FIG. 21C) of the single-channel, dual-end screw guide illustrated in FIG. 21A; and

FIG. 21D is a bottom view showing the first and second ends of the single-channel, dual-end screw guide of FIG. 21A coupled to a bone plate.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure.

The embodiments disclosed herein pertain to plating assemblies that include a screw guide with one or more pre-loadable bone screws (or other bone fastener(s)) and an attachable bone plate. The plating assemblies and screw guides herein provide a number of solutions to various challenges posed by current plating assemblies, particularly in the field of sternal closure, but also in various other bone plating indications. Many of these advantages pertain to ease of use. One such advantage provided by the screw guides herein is that they are pre-loadable with bone screws and readily attachable to an associated bone plate, which vastly improves screw handling and plate handling during a plating procedure. For example, surgeons or technicians need not pick and place bone screws onto a driver tip or manipulate low profile bone plates with their fingers or with forceps or other less-effective implements. Another advantage is that the screw guides herein are configured to couple interchangeably with a wide variety of plate shapes and configurations. An additional advantage is that the screw guides herein do not rely upon the interior plate hole threads to align the screws with the plate holes, thereby simplifying the screw insertion process. A further advantage is that the screw guides herein have geometries that allow a surgeon to manually use the screw guide itself as a counter-torque during screw insertion, thereby avoiding plate spin (e.g., “helicoptering”). Thus, the screw guides herein can be used to maintain the plate position during screw insertion without an assistant and/or other commonly used measures, such as partially inserting one or more screws through the plate holes to provisionally anchor the plate to the underlying bone prior to final screw fixation. Yet another advantage is that the screw guides herein provide sufficient manual leverage for a surgeon to press the bone plate flush against the bone without an assistant or additional instrument(s), such as forceps and the like that can be otherwise required.

Additional advantages provided by the screw guides herein pertain to the reduction/avoidance of particulate generation. Current screw guides commonly employ a press-fit (also termed an interference fit) between the screw head and the guide chamber to retain the bone screw therein prior to screw insertion through the associated plate hole. Such press-fit screw retention features can result in particulate generation as the screw is driven down the guide chamber toward the underlying plate hole. The screw guides herein reduce particulate generation by employing compliant screw retention mechanisms and/or other inventive retention features.

As used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although terms involving numerical prepositions (e.g., “first,” “second,” “third”) can be used herein to describe various features, such features should not be limited by these terms. These terms are instead used to distinguish one feature from another. For example, a first element could be termed a second element in another context, and, similarly, a second element could be termed a first element in another context, without departing from the scope of the embodiments disclosed herein.

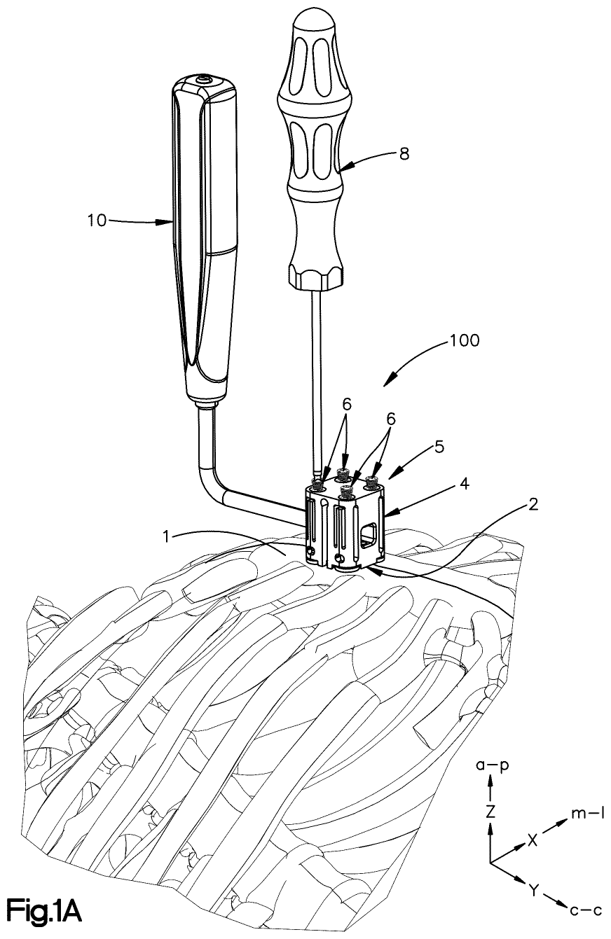

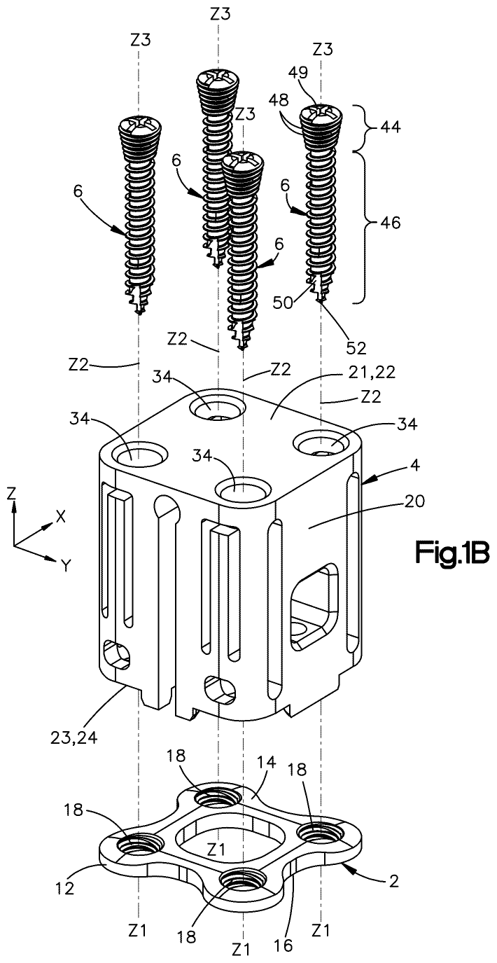

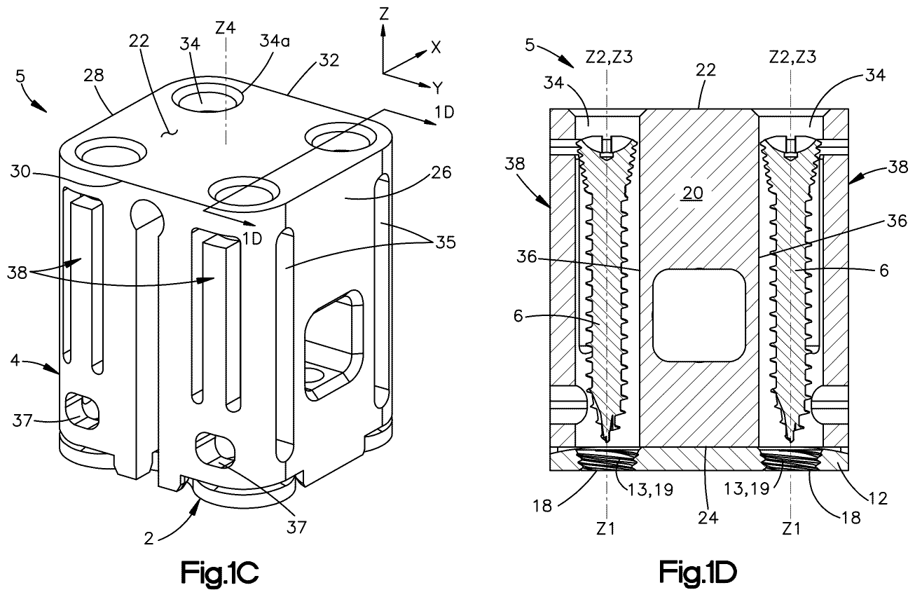

Referring to FIG. 1A, an exemplary bone plate system 100 is shown for affixing a bone plate to underlying bone 1. The bone plate system 100 includes a bone plate 2 and a fastener guide 4 (also referred to herein as a “guide” 4) that is attachable to the bone plate 2 and is configured to carry one or more pre-loaded fasteners 6 (e.g., bone screws) therein for affixing the bone plate 2 to the underlying bone 1. The guide 4, bone plate 2, and the fastener(s) 6 can be referred to collectively as a bone plating assembly 5. It should be appreciated that although the following reference to fasteners 6 refers primarily to bone screws 6, other types of fasteners can be employed, such as pins, nails, and the like. In the embodiments illustrated herein, the underlying bone 1 is the sternum and the bone plate system 100 is employed for sternal fixation, particularly for sternal closure following a sternotomy and/or sternal fracture. It should be appreciated, however, that the bone plate system 100 can be adapted for use plating other bones and providing other treatment types. Additionally, although FIG. 1A shows portions of the bone screws 6 extending proximally from the guide 4, it should be appreciated that such depiction is for visualization purposes and that the guide 4 is preferably configured such that the bone screws 6 reside entirely therein in their pre-loaded position.

The bone plate system 100 can include instrumentation, such as one or more driving tools 8 for driving the pre-loaded bone screws 6 through respective holes in the bone plate 2 and into underlying bone 1. The bone plate system 100 can also include an optional handle 10, which can be attachable to the guide 4 for providing enhanced manipulation of the guide 4 during a plating procedure. The handle 10 also allows provides the surgeon with a tool to provide counter-torque to the bone plate 2, via the guide 4 (and handle 10), during screw 6 insertion through and locking with the associated screw hole in the bone plate 2. The handle 10 and the guide 4 can have complimentary mounting structures for selective attachment and detachment of the handle 10 to and from the guide 4. For example, the handle 10 can include a male mounting structure configured to mate with a complimentary female mounting structure of the guide 4, or vice versa. Additionally or alternatively, the complimentary mounting formations of the handle 10 and the guide 4 can employ a ball and detent coupling mechanism for selective attachment and detachment of the handle 10 to and from the guide 4. Alternatively, the complimentary mounting formations can employ mating exterior and interior threads for threadably coupling and decoupling the handle 10 to and from the guide 4, respectively. It should be appreciated that various other types of complimentary mounting structures can be employed for attaching and detaching the handle 10 to and from the guide 4. In additional embodiments, the guide 4 can include a handle that is monolithic with the guide. It should also be appreciated that the guide 4 has an ergonomic design and can be manipulated by a surgeon during a plating procedure without the use of the handle 10.

Referring now to FIGS. 1B-1D, the bone plate 2 has a plate body 12 that defines an outer surface 14 and a bone-facing surface 16 opposite each other along a first direction Z. The plate body 12 defines one or more fastener holes 18 (e.g., screw hole(s) 18) each extending from the outer surface 14 to the bone-facing surface 16 along a respective central hole axis Z1. The one or more screw holes 18 can have internal threading 19 defined by an interior surface 13 of the plate body 12 with the hole(s) 18. As shown, the one or more screw holes 18 can include a plurality of screw holes 18 spaced from each other in a hole arrangement with respect to a second direction X and a third direction Y that are perpendicular to each other and to the first direction Z. The bone plate 2 of the illustrated embodiment is a non-contoured plate, although in other embodiments the bone plate 2 can be modified to include features for facilitating plate bending and/or contouring.

In the embodiments disclosed herein, the first direction Z generally aligns with an anterior-posterior direction a-p of patient anatomy; the second direction X generally aligns with one of a medial-lateral direction m−1 and a cranial-caudal direction c-c of patient anatomy; and the third direction Y generally aligns with the other of the medial-lateral and cranial-caudal directions m−1, c-c of patient anatomy. In the illustrated embodiments herein, the second direction X is shown aligned along, or alignable with, the medial-lateral direction m−1 of patient anatomy; and the third direction Y is shown aligned along, or alignable with, the cranial-caudal direction c-c of patient anatomy. It should be appreciated, however, that the second direction X can optionally be aligned along, or alignable with, the cranial-caudal direction c-c of patient anatomy; and the third direction Y can optionally be aligned along, or alignable with, the medial-lateral direction m−1 of patient anatomy. For purposes of discussion, the first direction Z can be referred to herein as a “vertical” direction and the second and third directions X, Y can be referred to herein as respective “horizontal” directions. Additionally, a plane extending along the second and third directions X, Y can be referred to herein as an “X-Y” plane or a “horizontal” plane. It should be appreciated that, when used with reference to an object herein, the foregoing directional terms (“vertical” and “horizontal”) and derivatives thereof (e.g., “vertically”, “upward”, “upper”, “top”, “downward”, “lower”, “bottom”, and “horizontally”) generally refer to the orientation(s) of such object(s) as illustrated in the Figures and are not to be considered as limiting the use of such objects to such orientations.

The guide 4 has a guide body 20 having a proximal end 21 and a distal end 23 spaced from each other along the first direction Z. The guide body 20 has a top surface 22 at the proximal end 21 and a bottom surface 24 at the distal end 23. The guide body 20 has a first side 26 and a second side 28 opposite each other along one of the second and third directions X, Y and a third side 30 and a fourth side 32 opposite each other along the other of the second and third directions X, Y. The guide body 20 also defines one or more guide barrels or “channels” 34 extending from the top surface 22 to the bottom surface 24 along a respective central channel axis Z2. As shown in FIG. 1D, each channel 34 is defined by an interior surface 36 of the guide body 20 and is configured to guide a respective bone fastener 6 (e.g., bone screw 6) into the respective plate hole 18. As shown in FIG. 1C, each channel 34 of the illustrated embodiment extends from an upper channel perimeter 34a at a boundary with the top surface 22 of the guide body 20. Each upper channel perimeter 34a preferably extends an entire revolution about the respective central channel axis Z2 in contiguous fashion with the top surface 22. Stated differently, each channel 34 preferably has an enclosed circumference at the top surface 22. The one or more channels 34 are each configured to carry a respective bone screw 6 therein in a pre-loaded position, which is in alignment with the one or more holes 18 of the bone plate 2. The guide 4 also includes a fastener retention mechanism 38 configured to retain the bone screw(s) 6 within the one or more channels 34 in the pre-loaded position, as described in more detail below. The fastener retention mechanism 38 can also be referred to as a “screw retention mechanism” 38 when used with bone screws 6, as in the embodiments illustrated herein. The guide body 20 also preferably includes a coupling mechanism 68 that facilitates coupling with the bone plate 2, as described in more detail below.

The guide body 20 also includes one or more visualization windows 35, 37 for providing visualization within the channel(s) 34, thereby allowing a surgeon to view the axial positions of the bone screws 6 therein. With reference to FIG. 1C, the guide body 20 of the illustrated embodiment defines vertically elongated visualization windows 35 extending into the channels 34, such as from the first and second sides 26, 28. The guide body 20 can also define lower visualization windows 37 extending into the channels 34, such as from the third and fourth sides 30, 32. The lower visualization windows 37 are configured to provide visualization of the plate holes 18, such as for viewing engagement of the bone screws 6 therein. It should be appreciated that various other visualization window configurations are within the scope of the present disclosure.

The distal end 23 of the guide body 20 is configured to couple with the plate body 12 at one or more guide orientations (i.e., orientations of the guide 4 relative to the bone plate 2), one such guide orientation being shown in FIGS. 1C-1D. The guide 4 is cooperatively configured with the bone plate 2 such that, when the guide 4 is attached to the bone plate 2 at such an orientation, the one or more channels 34 are aligned with the one or more plate holes 18 such that each central channel axis Z2 is substantially colinear with the associated central hole axis Z1. In the illustrated embodiment, the bone plate 2 has four (4) screw holes 18 spaced from each other in a square arrangement and the guide 4 has four (4) corresponding channels 34 spaced from each other in a square arrangement. In this manner, the guide 4 provides at least four (4) rotational guide orientations at which the guide 4 can couple with the bone plate 2 so that the channels 34 coaxially align with the plate holes 18, which rotational orientations can be measured about a central guide axis Z4 extending vertically through a geometric centerpoint between the four channels 34 (see FIG. 1C). It should be appreciated that other plate hole 18 arrangements and complimentary guide channel 34 arrangements are within the scope of the present disclosure, examples of some of which are described below.

The guide body 20 can be monolithic or, alternatively, can include two or more body components attached or otherwise jointed together. The guide body 20 can have a polymeric material composition, such as an injection-molded plastic, which can be transparent or semi-transparent for enhancing visualization of bone screws 6 therein. Alternatively, the guide body 20 can have a material composition that includes biocompatible metals or metal alloys (e.g., stainless steel, titanium), composites, foams, rubbers, and wood (treated and un-treated), by way of non-limiting examples. In one non-limiting example, the guide body 20 has a material composition that is stainless steel. The guide body 20 can be formed using various processes, including machining processes (e.g., mills, lathes, drill presses, bend presses), 3D printing or other rapid prototyping processes, casting, extrusion, forging, and blow molding, by way of non-limiting examples.

As shown, the bone fasteners 6 can be bone screws 6, which can each have a head 44 and a threaded shaft 46 extending therefrom along a fastener/screw axis Z3. The threaded shaft 46 is configured to advance through the associated plate hole 18 and into underlying bone for affixation therewith. In the illustrated embodiment, the bone screws 6 are locking head bone screws, wherein the screw heads 44 include external threading 48 configured to lock with the interior threading 19 of the plate holes 18 when the head 44 is fully seated in the hole 18. The screw heads 44 also define drive sockets 49 at the proximal ends thereof. The locking head bone screws 6 are preferably configured to lock within the associated plate hole 18 at a nominal orientation, at which the screw axis Z3 is substantially colinear with the central hole axis Z1. Thus, the external threading 48 of the screw head 44 and the internal threading 19 of the plate hole 18 preferably have complimentary thread geometries configured for nominal screw insertion. Additionally, the channels 34 of the guide 4 are preferably configured to facilitate screw insertion through the hole(s) 18 at nominal screw orientation. Accordingly, the channels 34 are preferably configured to carry and support the bone screws 6 therein such that the screw axes Z3 are substantially colinear with the central channel axes Z2 and central hole axes Z1. Thus, the interior surfaces 36 in the channels 34 are configured to guide the bone screws 6 into the plate holes 18 in substantially coaxial fashion. It should be appreciated that the threaded shafts 46 of the bone screws 6 can self-center the screws 6 relative to the plate holes 18 to an extent as the shaft threads contact the internal threading 19 of the plate holes 18 while the shafts 46 advance through the plate holes 18. It should be appreciated that the plate holes 18 and the channels 34 can be adapted for use with variable angle locking (VAL) bone screws for insertion and locking with the plate holes 18 at nominal screw orientations. The bone screws 6 are preferably self-drilling, as shown, having distal cutting flutes 50 that extend distally to the distal screw tip 52 for self-drilling into and through the underlying bone. In other embodiments, the bone screws 6 can be self-tapping screws for use with pilot holes. In such other embodiments, the guide 4 can facilitate pre-drilling through one or more of the channels 34, after which the screws 6 can be loaded into the channels 34.

Referring now to FIGS. 1E-1F, the screw retention mechanism 38 of the guide 4 will now be described. The screw retention mechanism 38 can include at least one compliant member 40 that extends within the channel 34 and is configured to contact the screw 6 and apply a retention force thereto sufficient to retain the screw within the channel 34 in the pre-loaded position at least against the force of gravity. Stated differently, the at least one compliant member 40 is configured to apply the retention force to the screw 6 at a magnitude that is at least sufficient to prevent the screw 6 from dropping through the channel 34 and into the plate hole 18 responsive to gravity. The screw retention mechanism 38 is preferably configured such that the retention force is also sufficient to hold the screw 6 in the pre-loaded position while the surgeon couples the driver to the drive socket of the screw head 44 and subsequently allowing the screw 6 to decouple from the screw retention mechanism 38 responsive to a threshold driving force applied to the screw 6 along the screw axis Z3.

In the illustrated embodiment, each channel 34 has at least one respective compliant member 40 that extends within the channel 34 and provides the retention force. The compliant members 40 are each a defined by the guide body 20. The compliant member 40 can be a flexible tab 40 defined by a cutout portion 41 of the guide body 20. The cutout portion 41 can have an inverted U-shape such that the flexible tab 40 extends upwardly from a first, connected tab end 43 to a second, free tab end 45, which is positioned below the top surface 22 of the guide body 20. In other embodiments, the flexible tabs 40 can extend downwardly (toward the bottom surface 24 of the guide body 20) from the connected ends 43 to the free ends 45, as described below. In yet other embodiments, the flexible tabs 40 can extend along other directions from their connected ends 43 to their free ends 45, such as along directions having at least a directional component along the second and/or third direction X, Y. Other flexible tab 40 configurations are also within the scope of the present disclosure. It should be appreciated that the cutouts 41 can effectively define additional visualization windows for viewing the bone screws 6 within the channels 34.

As best shown in FIG. 1F, the flexible tabs 40 are bent or otherwise formed so that interior surfaces 47 of the tabs 40 at the free ends 45 thereof extend inwardly into the respective channels 34 a distance sufficient to contact the associated screw head 44 and apply the retention force thereto for maintaining the screws 6 in the pre-loaded position. The flexible tabs 40 are configured to flex outwardly responsive to a threshold driving force applied to the screw 6 toward the plate hole 18, thereby allowing the screw head 44 to decouple from the flexible tab 40 when the surgeon begins driving the screw 6 along the screw axis Z3. In the illustrated embodiment, each channel 34 has an associated flexible tab 40 that extends inwardly to the channel 34 from the third or fourth side 30, 32 of the guide body 20. In other embodiments, the flexible tabs 40 can be defined along the first and second sides 26, 28 of the guide body 20. It should be appreciated that the flexible tabs 40 can eliminate the need to employ a rigid interference fit for retaining the screws 6 within the channels 34, thereby avoiding or at least reducing the particulates generated by the use of such interference fits for screw retention within a guide. It should also be appreciated that the flexible tabs 40 can have various alternative configurations without departing from the scope of the present disclosure. It should further be appreciated that the flexible tabs 40 can be adapted as needed to provide different retention force magnitudes.

It should also be appreciated that various other screw retention mechanism configurations are within the scope of the present disclosure, some additional examples of which are described below.

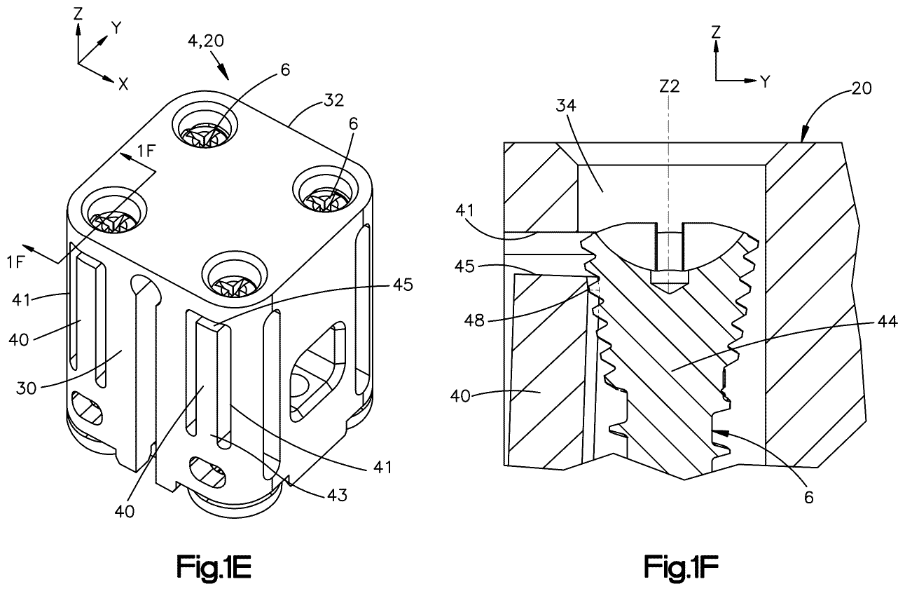

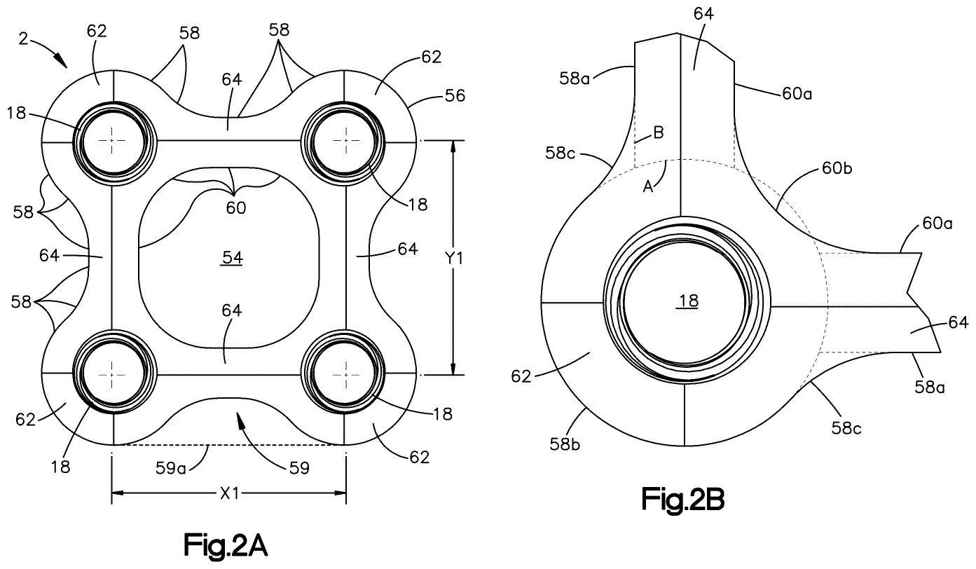

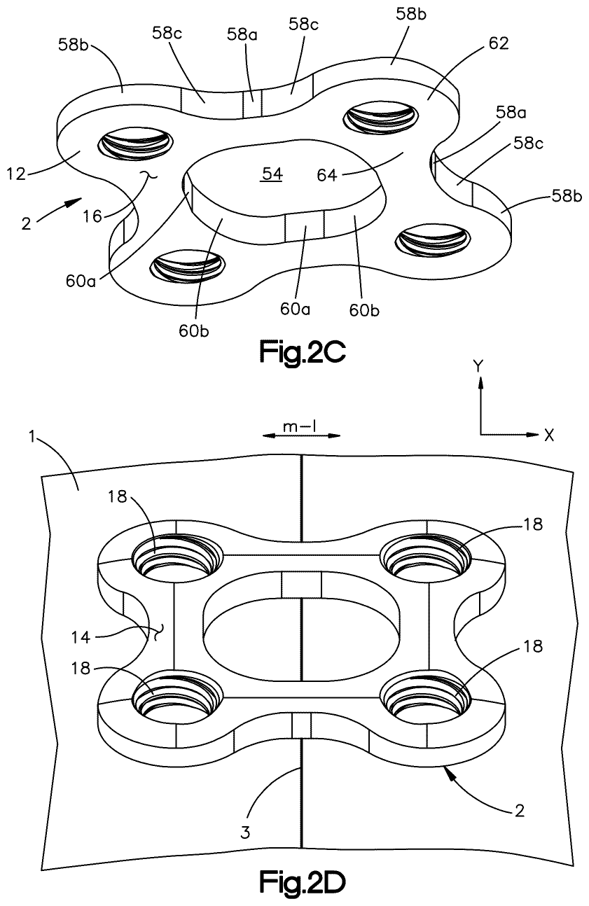

Referring now to FIGS. 2A-2D, the bone plate 2 has a geometry configured to facilitate easy coupling with and decoupling from the guide 4. In the illustrated embodiment, the plate body 12 defines an aperture 54 that extends from the outer surface 14 to the bone-facing surface 16 along the first direction Z. The aperture 54 can be centrally located in the plate body 12 with respect to the second and third directions X, Y, as shown, although in other embodiments the aperture 54 need not be centrally located. An outer periphery 56 of the plate body 12 has exterior side surfaces 58 extending between the outer surface 14 and the bone-facing surface 16 along the first direction Z. The plate body 12 also defines interior side surfaces 60 located within the aperture 54 and extending between the outer surface 14 and the bone-facing surface 16 along the first direction Z. In the illustrated embodiment, the outer surface 14 is contiguous with (i.e., shares a common boundary with) the exterior and interior side surfaces 58, 60 of the plate body 12, which are, in turn, contiguous with the bone-facing surface 16 of the plate body 12. In other embodiments, however, the plate body 12 can include one or more additional surfaces, such as relief surfaces (e.g., chamfer surfaces), which can extend between the outer surface 14 and any of the exterior and/or interior side surfaces 58, 60, and/or between the bone-facing surface 16 and any of the exterior and/or interior side surfaces 58, 60. Examples of bone plates having such additional relief surfaces are described below.

In the illustrated embodiment, the plate body 12 has four plate holes 18 arranged in an equilateral rectangle (i.e., a square) arrangement, which provides the holes 18 with equidistant hole spacings X1, Y1 along the first and second directions X, Y, respectively. This hole spacing configuration is particularly beneficial in the context of sternal closure fixation because it allows the bone plate 2 to span the incision 3 of the sternum 1 at multiple plate orientations along an X-Y reference plane, each with two pairs of the holes 18 spaced from each other along the medial-lateral direction m−1 of patient anatomy (see FIG. 2D) in bracket-like fashion. Stated differently, the bone plate 2 according to the illustrated embodiment can be oriented relative to the sternum 1 such that either the second direction X or the third direction Y of the bone plate 2 is oriented along the medial-lateral direction m−1, with two (2) holes 18 on each side of the incision 3. In other embodiments, however, the bone plate can have non-equidistant hole spacings X1, Y1, as described in more detail below.

Additionally, the plate body 12 of the illustrated embodiment has node portions 62 (also referred to herein as “nodes” 62) in which the holes 18 are defined and bridge portions 64 (also referred to herein as “bridges” 64) that interconnect the nodes 62. As shown, the nodes 62 can have a generally circular shape and the bridges 64 can have a generally rectangular shape in an X-Y reference plane, as indicated by dashed lines A, B shown in FIG. 2B. The presence of the nodes 62 and bridges 64 provides the exterior side surfaces 58 and the interior side surfaces 60 of the plate body 12 with generally undulating profiles in the X-Y reference plane, the profiles having protrusions along the nodes 62 and recesses 59 along the bridges 64, which can facilitate coupling with the guide 4. For illustrative purposes, one such recess 59 along an exterior side surface 58 of the plate body 12 is shown in FIG. 2A being inwardly recessed from a reference line 59a that intersects extremities of the adjacent nodes 62. As shown in FIGS. 2B-2C, the exterior side surfaces 58 of the plate body 12 include exterior bridge side surfaces 58a and exterior node side surfaces 58b, while the interior side surfaces 60 (within the aperture 54) include interior bridge side surfaces 60a and interior relief side surfaces 60b, the latter of which are located at intersections between the nodes 62 and bridges 64 within the aperture 54 (i.e., at corners of the aperture 54). Preferably, the exterior side surfaces 58 of the plate body 12 also include exterior side relief surfaces 58c at intersections between the nodes 62 and bridges 64. The exterior and interior relief surfaces 58c, 60b beneficially reduce stress concentrations in the plate body 12.

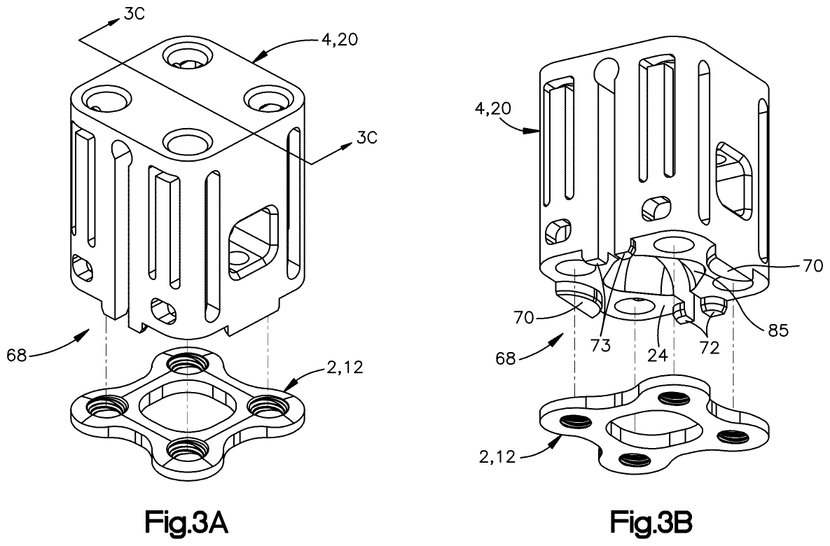

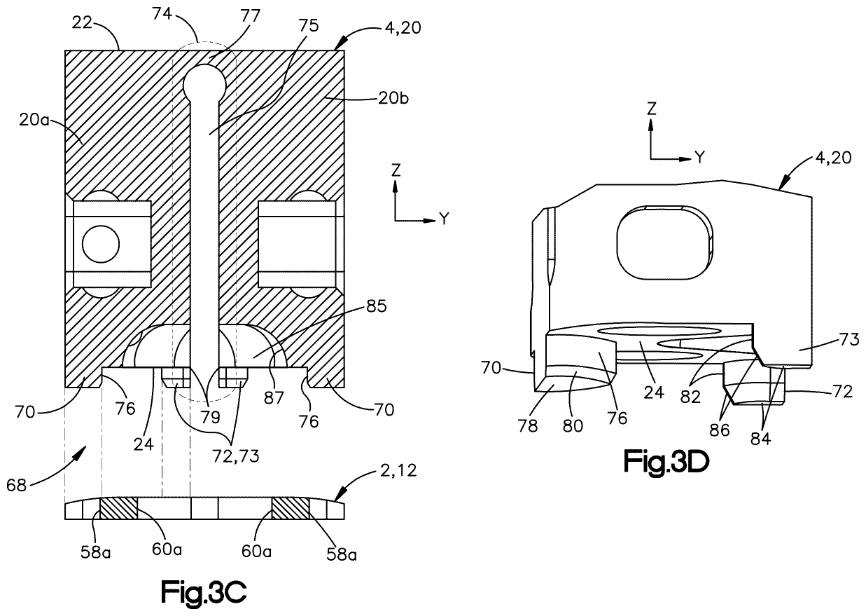

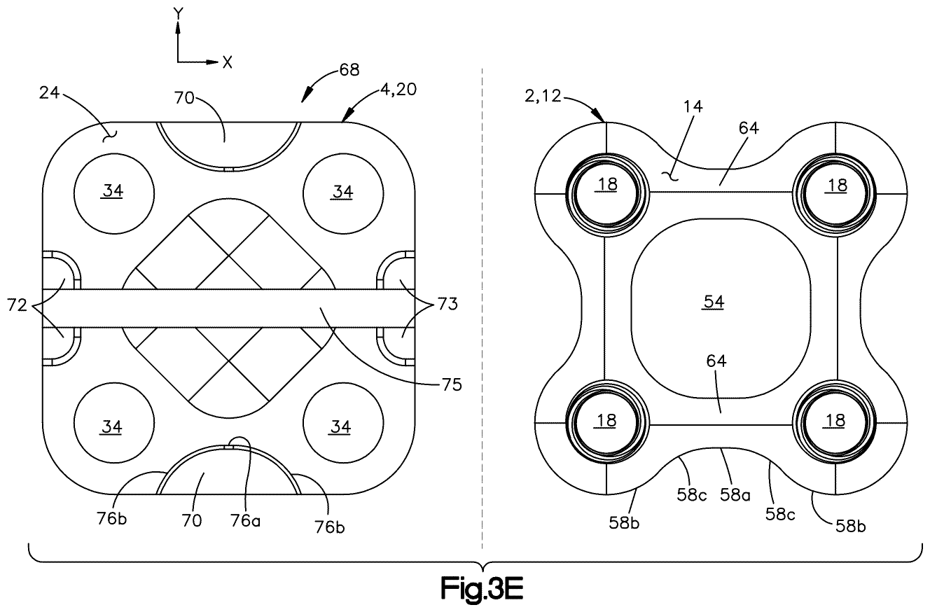

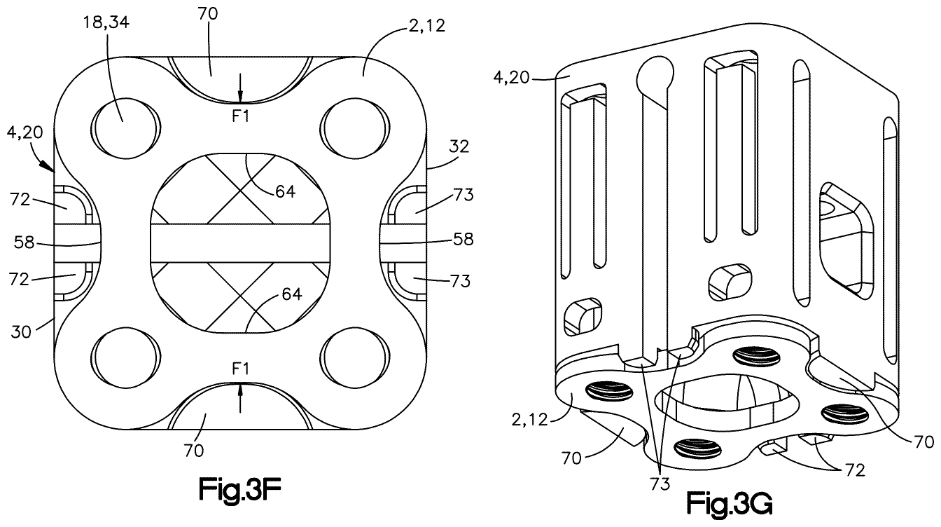

One benefit of the bone plate 2 design of the illustrated embodiment is that the bone plate 2 effectively forms a mounting bracket for coupling with the guide 4. Referring now to FIGS. 3A-3G, coupling between the guide 4 and the bone plate 2 will now be described. The guide body 20 includes a coupling mechanism 68 that facilitates coupling with the bone plate 2 at one or more guide orientations. The coupling mechanism 68 is configured to grip the bone plate 2 with a coupling force that is at least sufficient to maintain coupling engagement against the force of gravity and preferably greater, though not so great to cause difficulty decoupling the guide 4 after screw insertion and fixation with bone. For example, in some embodiments, the coupling force can be in a range of about 10 Newtons (N) to about 150 Newtons (N), although the coupling mechanisms can be adapted for coupling forces outside the aforementioned range. The coupling mechanism 68 of the illustrated embodiment includes a plurality of engagement members 70 that protrude downwardly from the bottom surface 24 of the plate body 12 along the first direction Z. The engagement members 70 are configured to engage and grip associated portions of the plate body 12.

The coupling mechanism 68 also includes a spring member 74 configured to bias one or more of the engagement members 70 into engagement with the associated portions of the plate body 12. In the illustrated embodiment, the spring member 74 includes a spring relief slot 75 defined in the guide body 20. The slot 75 extends from the bottom surface 24 toward the top surface 22 along the first direction Z. The slot 75 also extends from the third side 30 to the fourth side 32 of the guide body 20 along the second direction X. The slot 75 effectively separates the guide body 20 into a first guide body portion 20a and a second guide body portion 20b located on opposite sides of the slot 75 along the third direction Y. The first and second guide body portions 20a,b are connected by a neck 77 of the guide body 20 that extends along the first direction Z from an upper end of the slot 75 to the top surface 22 of the guide body 20. The neck 77 provides a measure of flexibility between the first and second guide body portions 20a,b along the third direction Y, particularly at lower ends 79 the first and second guide body portions 20a,b.

In the illustrated embodiment, the engagement members 70 include a first pair of engagement members 70 that are configured to engage one or more of the exterior side surfaces 58 of the plate body 12 in a manner providing a clamping or compressive force for coupling the guide 4 with the bone plate 2. Accordingly, one of the engagement members 70 is located on one side of the slot 75 and the other engagement member is located on the other side of the slot 75.

The engagement members 70 each have an interior surface 76 that extends downward from the bottom surface 24 of the guide body 20 toward a bottom surface 78 of the engagement member 70. The engagement members 70 also include at least one chamfer surface 80 extending between the interior surfaces 76 and the bottom surfaces 80. The interior surfaces 76 of the engagement members 70 are configured to engage associated exterior side surfaces 58 of the plate body 12 to apply the coupling force thereto. In particular, in the illustrated embodiment, the interior surfaces 76 of the engagement members 70 are configured to engage at least against opposite exterior bridge surfaces 58a along the third direction Y. Accordingly, as shown in FIG. 3E, each interior surface 76 includes a first surface portion 76a configured to engage one of the exterior bridge side surfaces 58a. The first surface portions 76a face each other in the illustrated embodiment. The interior surfaces 76 can also include one or more second surface portions 76b configured to engage one or more of the exterior relief surfaces 58c and/or exterior node side surfaces 58b.

As shown in FIGS. 3B-3C, the coupling mechanism 68 can include one or more additional engagement members, such as a second pair of engagement members 72 and a third pair of engagement members 73, which can each be spaced intermediate the first pair of engagement members 70 with respect to the third direction Y. For example, the second and third pairs of engagement members 72, 73 can be spaced from each other along the second direction X. The engagement members 72 of the second pair can be spaced from each other on opposite sides of the slot 75 along the third direction Y. Similarly, the engagement members 73 of the third pair can be spaced from each other on opposite sides of the slot 75 along the third direction Y. The second and third pairs of engagement members 72, 73 of the illustrated embodiment are configured to extend alongside opposed exterior side surfaces 58 of the plate body, particularly alongside respective exterior side relief surfaces 58c. The second and third pairs of engagement members 72, 73 can be configured to provide horizontal support to the bone plate 2 and/or to center the guide 4 in the desired orientation relative to the bone plate 2. As shown in FIG. 3D, each of the second and third pairs of engagement members 72, 73 can have an interior surface 82 that extends downward from the bottom surface 24 of the guide body 20 toward a bottom surface 84 of the respective engagement member 72, 73. The second and third pairs of engagement members 72, 73 can each also include at least one chamfer surface 86 extending between the respective interior surface 82 and the respective bottom surface 84.

Referring now to FIGS. 3E-3G, to couple the guide 4 to the bone plate 2, at least one of the bottom surface 24 of the guide 4 and the outer surface 14 of the bone plate 2 is brought toward the other (or they can be brought toward each other), along the first direction Z, at the orientation shown in FIGS. 3A-3B. The at least one chamfer surfaces 80 of the first pair of engagement members 70 contact the outer surface 14, or an interface (e.g., edge) between the outer surface 14 and the exterior side surfaces 58 of the plate body 12. As the bottom surface 24 of the guide 4 and the outer surface 14 of the bone plate 2 are brought closer together along the first direction Z, the engagement between the chamfer surfaces 80 and the plate body 12 pushes the lower ends 79 of the first and second guide body portions 20a,b away from each other along the third direction Y, causing the spring member 74 at the neck 77 to provide an opposite, inward bias force F1 along the third direction Y (FIG. 3F). As the interior surfaces 76 of the first pair of engagement members 70 engage the exterior side surfaces 58 of the plate body 12, this inward bias force F1 presses the interior surfaces 76 against the exterior side surfaces 58, including the first surface portions 76a against the interfacing exterior bridge side surfaces 58a, with sufficient force to initiate and maintain coupling between the guide 4 and the bone plate 2. Additionally, engagement of the second surface portions 76b of the first pair of engagement members 70 against the exterior node and relief side surfaces 58b,c can provide a centering function that helps maintain alignment of the guide at the desired orientation relative to the bone plate 2 (i.e., so that the plate holes 18 are aligned with the channels 34 of the guide 4), as shown in FIG. 3F. Additionally, during coupling, engagement of the interior surfaces 82 of the second and third pairs of engagement members 72, 73 with the exterior node and relief side surfaces 58b,c of the plate body 12 can also facilitate centering and/or retaining of the guide 4 at the desired orientation relative to the bone plate 2.

The guide body 20 can also include one or more structures for facilitating decoupling of the bone plate 2 and the guide 4 from one another. For example, as best shown in FIGS. 3B-3C, the guide body 20 of the illustrated embodiment defines a decoupling structure in the form of a recess 85 configured for abortive decoupling (e.g., pre-operative or intra-operative decoupling) of the bone plate 2 and guide 4 from one another. Within the recess 85, the guide body 20 defines a recessed surface 87 that extends from the bottom surface 24 toward the top surface 22 (see FIGS. 3B-3C) and is configured to receive a portion of an instrument for decoupling the guide 4 from the bone plate 2. The recessed surface 87 has a concave, generally dome-like profile (FIG. 3C) that spans the slot 75 and can be positioned symmetrically in the first and second guide body portions 20a,b. As shown, the recess 85 can be centrally located in the bottom surface 24 with respect to the second and third directions X, Y. Additionally, the recess 85 is preferably configured to align with the aperture 54 of the bone plate 2 when the guide 4 and bone plate 2 are coupled. In this manner, when the guide 4 and bone plate 2 are coupled, a decoupling instrument can be inserted through the aperture 54 and into the recess 85 from underneath the bone plate 2 and engaged against the recessed surface 87 in a manner that facilitates decoupling the guide 4 from the bone plate 2. By way of a non-limiting example, the decoupling instrument can be pliers, which can be employed so that the jaws thereof engage the recessed surface 87. At such an engaged position, the jaws can be expanded against portions of the recessed surface 87 on opposite sides of the slot 75 to impart an outward decoupling force (along the third direction Y in this example) against the first and second guide body portions 20a,b that counteracts the bias force F1 and facilitates decoupling the bone plate 2 and the guide 4 from one another. Additionally or alternatively, the decoupling instrument can impart a decoupling force against the recessed surface 87 in a direction toward the proximal end 21 of the guide body 20. It should be appreciated that the recess 85 and recessed surface 87 described above are provided as one example of a decoupling structure(s) of the guide 4, while various other geometries and configurations for decoupling structures are within the scope of the embodiments herein.

It should be appreciated that, in other embodiments, the positions of the first pair of engagement members 70 and the recess 85 can be switched with each other. For example, the first pair of engagement members 70 can be positioned inwardly of a pair of recesses 85 with respect to the third direction Y, such that the first pair of engagement members 70 are configured to extend within the aperture 54 of the bone plate 2 when coupled thereto. In this example, the first pair of engagement members 70 can be configured to provide an outward bias force F1 against interior side surfaces 60 of the plate body 12 within the aperture 54, thereby clamping the guide 4 to the bone plate 2. Furthermore, in this example, the guide body 20 defines a pair of decoupling recesses 85 that are spaced outwardly from the engagement members 70 and can be aligned with respective recesses 59 along the exterior side surfaces 58 of the plate body 12. It should further be appreciated that yet other configurations for the engagement members 70 and/or the decoupling recess(es) 85 are within the scope of the present disclosure. Additional example coupling mechanisms for coupling the guide 4 to the bone plate 2 are described below.

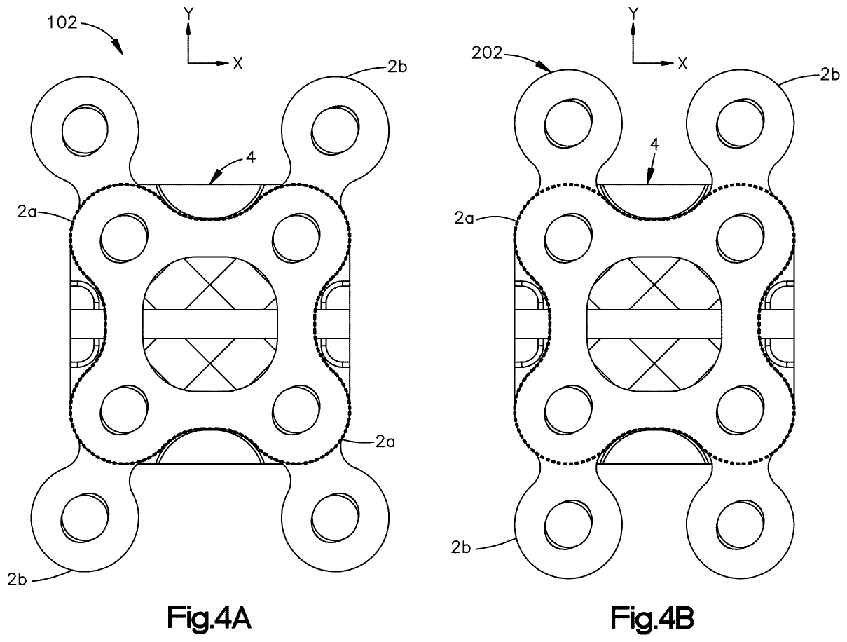

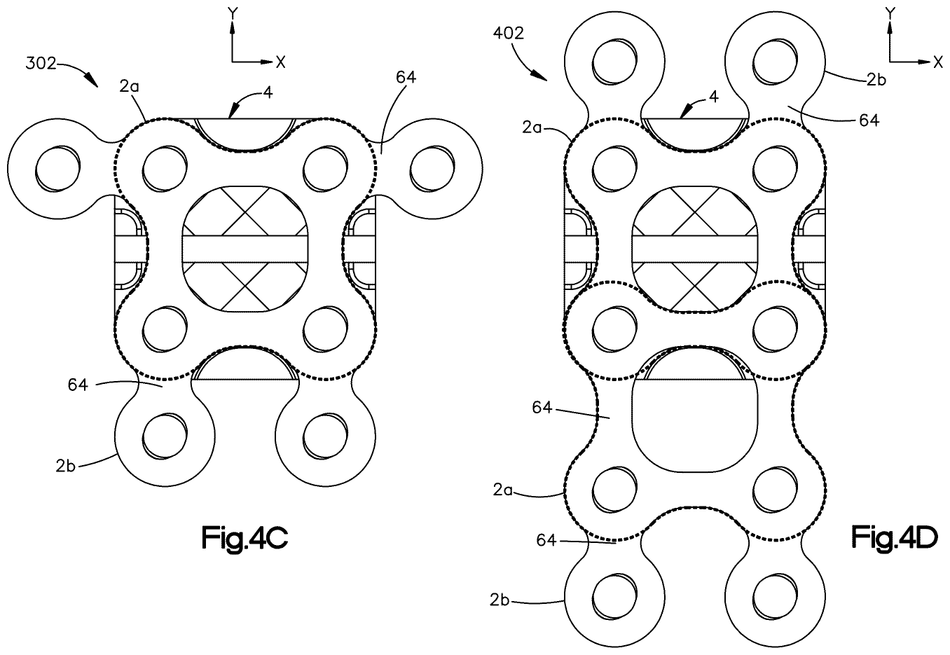

Referring now to FIGS. 4A-4D, additional example bone plates 102, 202, 302, 402 are shown that employ the design of the bone plate 2 described above as a hub or bracket portion 2a from which additional plate portions, such as plate arms 2b, can extend, thereby increasing the plate coverage area for underlying bone and providing additional plate holes 18. Thus, the bone plates 102, 202, 302, 402 of the present embodiments can be referred to as “extended-area” bone plates. For example, FIG. 4A shows an example “X-plate” style bone plate 102 having four (4) arms 2b extending from the hub portion 2a at respective oblique angles, the bone plate 102 providing a total of eight (8) holes. FIG. 4B shows an example “H-plate” style bone plate 202 having four arms 2b extending from the hub portion 2a along parallel directions, the bone plate 202 providing a total of eight (8) holes. FIG. 4C shows an example “double T-plate” style bone plate 302 (also referred to as a “pi” plate) having four (4) arms 2b extending from the hub portion 2a, two (2) of the arms 2b extending colinearly along one of the second or third directions X, Y, and two (2) of the arms 2b extending in parallel fashion along the other of the second and third directions X, Y. The bone plate 302 in this example provides a total of eight (8) holes 18. FIG. 4D shows an example “ladder plate” style bone plate 402, effectively having two (2) hub portions 2a in sequence along the second direction X and sharing a common bridge 64 (and its associated pair of holes 18), each of the hub portions 2a having a pair of arms 2b extending outwardly therefrom in parallel fashion along the second direction X. The bone plate 402 in this example provides a total of ten (10) holes 18. It should be appreciated that, for this example bone plate 402, the guide 4 can couple to either of the hub portions 2a.

The foregoing exemplary extended-area bone plates 102, 202, 302, 402 can be employed for sternal closure, such as on the sternal manubrium and/or the sternal body, for example. Additional sternal bone plate types and designs can employ the hub portion 2a described above, including T-plates, L-plates, angled plates, and straight plates (for affixation to various ribs), by way of non-limiting examples. It should also be appreciated that the hub portion 2a can be employed on numerous types, styles, and sizes of bone plates for indications other than sternal fixation.

It should be appreciated that the hub portion 2a can effectively provide a universal coupling structure that can be employed in a plurality of plate designs for coupling interchangeably with a single screw guide or class of screw guides that employ a complimentary coupling mechanism. In the illustrated embodiments, the hub portion 2a is centrally located in the X-plate 102 and the H-plate 202 with respect to each of the second and third directions X, Y. In other embodiments, the hub portion 2a need not be centrally located with respect to one or both of the second and third directions X, Y. For example, in the double-T plate 302 shown in FIG. 4C and in the ladder plate 402 shown in FIG. 4D, the hub portions 2a thereof are shown non-centrally located with respect to the third direction Y. In further embodiments, an extended-area bone plate, such as the ladder plate 402 shown in FIG. 4D, can have two or more hub portions 2a, each couplable with a respective screw guide. As shown in the foregoing example extended area bone plates 102, 202, 302, 402, the plate arms 2b preferably have node 62 and bridge 64 portions similar to those described above with reference to the bone plate 2 shown in FIGS. 2A-2D, which node 62 and bridge 64 portions can facilitate coupling with additional screw guides, such as single-channel screw guides designed for screw insertion along the plate arms 2b. Examples of single-channel screw guides are described in more detail below.

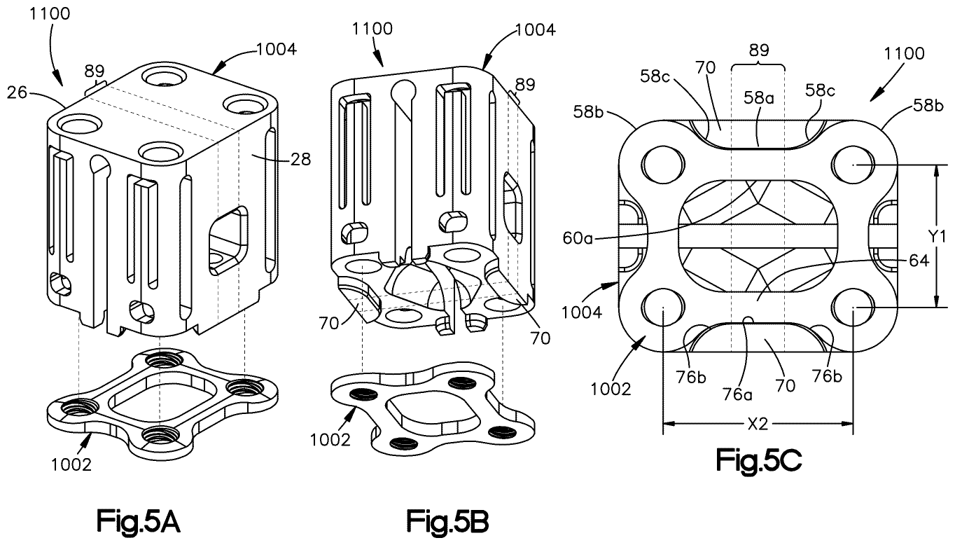

Referring now to FIGS. 5A-5C, in additional embodiments, a bone plate system 1100 can employ a bone plate 1002 and an associated guide 1004 that have non-equidistant hole spacings X2, Y1 and channel spacings, respectively. For example, the bone plate 1002 of such a bone plate system 1100 can be substantially similar to the bone plate 2 described above with reference to FIGS. 2A-2D, but with a hole spacing X2 along the second direction X that is greater than the hole spacing Y1 along the third direction Y. The increased hole spacing X2 along the second direction X can be achieved by increasing the lengths of the bridges 64 that extend along the second direction X, while maintaining the other portions of the bone plate 1002 substantially similar to those of bone plate 2 described above. Accordingly, bone plate 1002 can be referred to as a “wide” or “large” version of bone plate 2. Similarly, the guide 1004 associated with bone plate 1002 can be substantially similar to the guide 4 described above, but having the first and second sides 26, 28 of the guide body 20 lengthened along the second direction X. Accordingly, the guide 1004 of the present embodiment can be referred to as a “large” version of guide 4, or otherwise referred to as a “large guide” 1004. In the illustrated example, the portion of the guide body 20 that is lengthened along the second direction X is the portion 89 located between the lateral extremities of the first surface portions 76a of the first pair of engagement members 70, which lengthened portion 89 is indicated by dashed lines. Additionally, the portion of the bone plate 1002 that is lengthened along the second direction X can be the portion(s) of the respective bridges 64 that define the exterior and/or interior side surfaces 58a, 60a thereof.

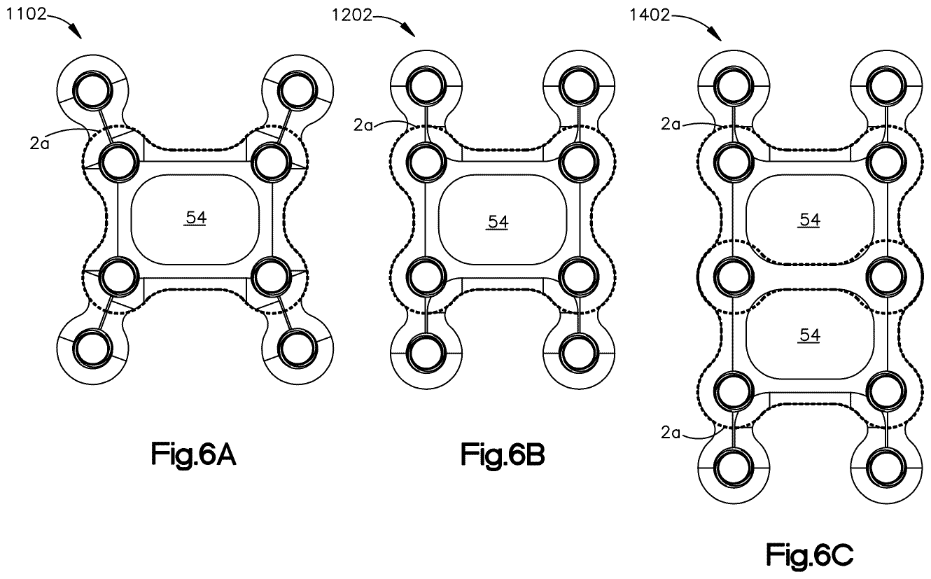

Referring now to FIGS. 6A-6C, additional exemplary bone plates 1102, 1202, 1402 are shown that employ the “large” bone plate 1002 design as a hub portion 1002a. These example bone plates 1102, 1202, 1402 are otherwise similar to bone plates 102, 202, 402 described above, respectively, and can thus be referred to as respective “wide” or “large” versions of bone plates 102, 202, 402. In particular, the bone plate 1102 shown in FIG. 6A can be referred to a “large X-plate”; the bone plate 1202 shown in FIG. 6B can be referred to a “large H-plate”; and the bone plate 1402 shown in FIG. 6C can be referred to a “large ladder plate”. Although not shown, a large version of the “double T-plate” style bone plate 302 shown in FIG. 4C is also within the scope of the present disclosure. It should be appreciated that each of the large bone plates 1102, 1202, 1402 can be coupled interchangeably with the large guide 1004 described above with reference to FIGS. 5A-5C. In such uses, the guide 1004 can be used to insert fasteners (e.g., screws) through the plate holes 18 of the hub portion 2a of the bone plate 1102, 1202, 1402, whereas the plate holes 18 of the arm portions 2b of the bone plate 1102, 1202, 1402 can receive fasteners therein via free-hand or via single-barrel guides, examples of which are described in more detail below.

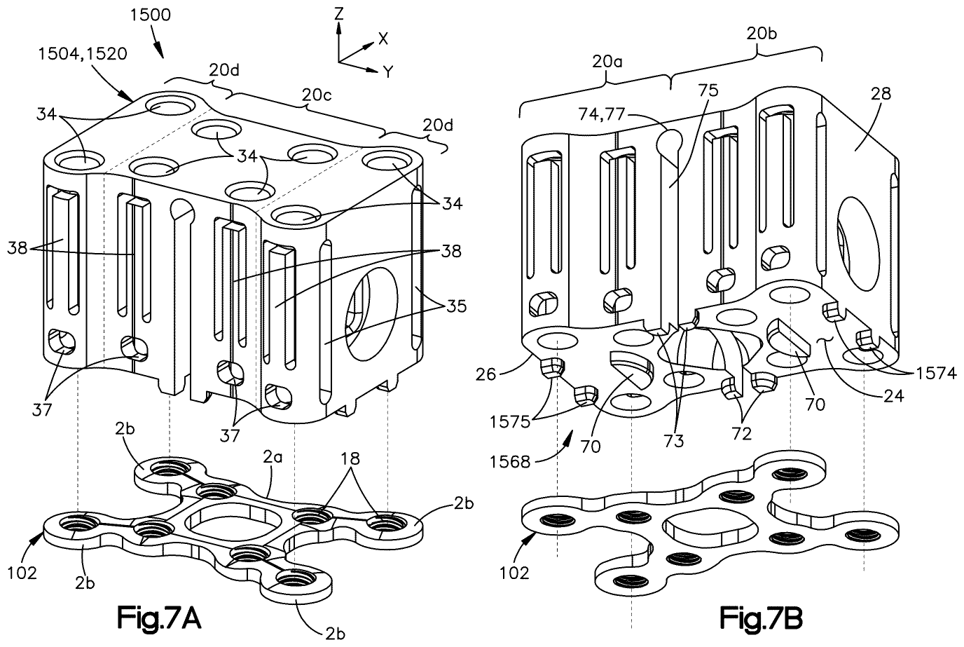

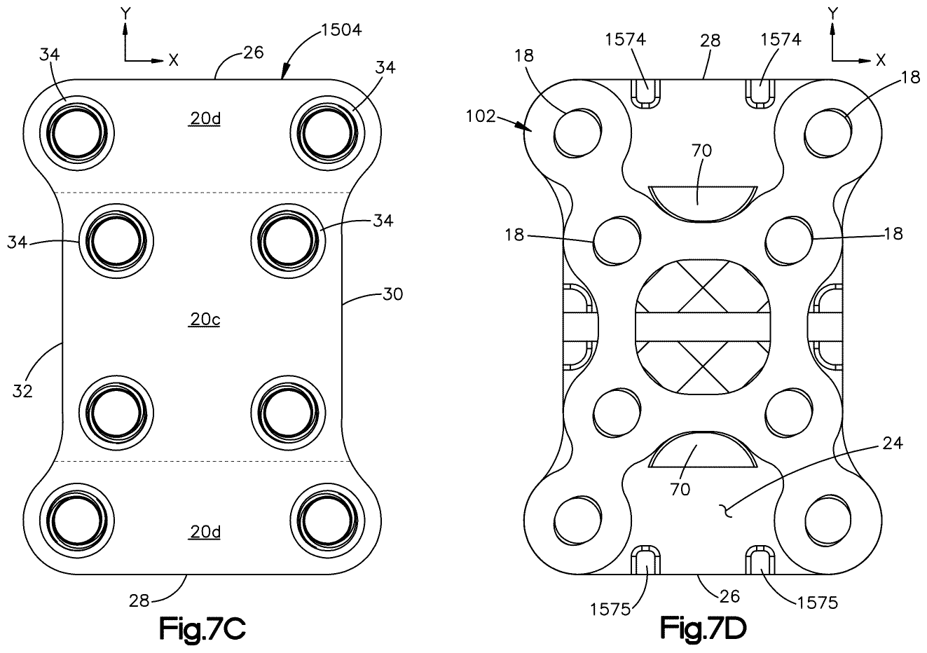

With reference to FIGS. 7A-9F, additional embodiments of exemplary bone plate systems 1500, 1600, 1700 that include extended-area guides 1504, 1604, 1704 for use with one or more extended-area bone plates will now be described. The extended-area guides of these example embodiments can be generally similar to the guides 4, 104 described above. For the sake of brevity, the following discussion focuses on some of the differences employed in the screw guides 1504, 1604, 1704 of FIGS. 7A-9F relative to the screw guides 4, 104 described above with reference to FIGS. 1A-5C. Furthermore, in the following discussion, features of the extended-area fastener guides 1504, 1604, 1704 that have similar design and function to those fastener guides 4, 104 described above can employ the same reference numbers.

Referring now to FIGS. 7A-7D, an exemplary bone plate system 1500 includes an extended-area guide 1504 configured to couple with, and decouple from, an extended-area bone plate, particularly the X-plate style bone plate 102 described above with reference to FIG. 4A. Accordingly, the extended-area guide 1504 of this embodiment can be referred to as an “X-guide” 1504. The X-guide 1504 has a guide body 1520 that defines a plurality of channels 34, which preferably correspond to and align with each of the plate holes 18 of the bone plate 102 when the guide 1504 and bone plate 102 are coupled together in a preferred orientation relative to each other, such as the orientation shown in FIGS. 7A-7D. As shown, the guide body 1520 can have a central body portion 20c that aligns with the hub portion 2a of the bone plate 102 and can be configured substantially similar to the guide 4 described above. The guide body 1520 also has a pair of extended body portions 20d that are configured to align with the arms 2b of the bone plate 102. The central body portion 20c of this example is positioned intermediate the extended body portions 20d along the third direction Y. The extended body portions 20d can define the first and second sides 26, 28 of the X-guide 1504, which in the present embodiment can be referred to as first and second “ends” of the guide 1504.

The central body portion 20c in this example defines four (4) channels 34 that are arranged in a square pattern and are configured to extend coaxially with the plate holes 18 of the hub portion 2a, respectively, when coupled. Each of the extended body portions 20d defines a pair of channels 34 that are configured to extend coaxially with the plate holes 18 of the plate arms 2b, respectively, when coupled. Accordingly, the third and fourth sides 30, 32 of the X-guide 1504 can flare outwardly along the second direction X as they extend from the central body portion 20c to and along the extended body portions 20d. Preferably, each of the channels 34 of the extended body portions 20d include a screw retention mechanism 38, which can each be similar to those described above. Additionally, each of the channels 34 of the extended body portions 20d can be open to one or more visualization windows 35, 37, which can be similar to those described above. The central body portion 20c of this example defines the spring member 74, the spring relief slot 75, and the neck 77, which provides the inward bias force F1 to first and second portions 20a,b of the guide body 1520, similarly as described above. The first portion 20a of the guide body 1520 extends from the neck 77 to the first end 26, and the second portion 20b of the guide body 1520 extends from the neck 77 to the second end 28. Thus, in the present example, the first and second portions 20a,b of the guide body 1520 both include about one half of the central body portion 2c and one of the pair of extended body portions 20d.

The X-guide 1504 includes a coupling mechanism 1568, which can include the first engagement members 70 and the second and third pairs of engagement members 72, 73 described above. The coupling mechanism 1568 of the present embodiment can also include additional engagement members, which can be located at or adjacent the first and second ends 26, 28 of the X-guide 1504, and can be configured for engaging the arms 2b of the bone plate 102 to facilitate centering and/or retaining of the guide 1504 at the desired orientation relative to the bone plate 102. For example, such additional engagement members can include an optional fourth pair of engagement members 1574 located at the first end 26 of the guide body 1520 and an optional fifth pair of engagement members 1575 located at the second end 28 of the guide body 1520. The fourth and fifth pairs of engagement members 1574, 1575 each extend downwardly from the bottom surface 24 of the guide body 1520. Within each of the fourth and fifth pairs of engagement members 1574, 1575, the engagement members thereof are spaced from each other such that, when the X-guide 1504 and the bone plate 102 are coupled in the preferred orientation, the engagement members 1574, 1575 are received within a space between opposed arms 2b of the bone plate 102 along the second direction X. Preferably, the fourth and fifth pairs of engagement members 1574, 1575 are configured such that each respective fourth and fifth pair extend alongside, and in close proximity to or engagement with, the respective opposed exterior side surfaces 58b of the plate arms 2b along the second direction X. In this manner, the fourth and fifth pairs of engagement members 1574, 1575 can help inhibit or reduce toggling motion of the bone plate 102 when coupled with the X-guide 1504 in the preferred orientation. In this regard, the fourth and fifth pairs of engagement members 1574, 1575 can supplement the centering and/or retaining functionality of the X-guide 1504 and bone plate 102 provided by the first, second, and third pairs of engagement members 70, 72, 73. It should be appreciated that various other configurations and geometries for additional engagement members (e.g., in addition or alternative to the fourth and fifth pairs of engagement members 1574, 1575) are within the scope of the present embodiment.

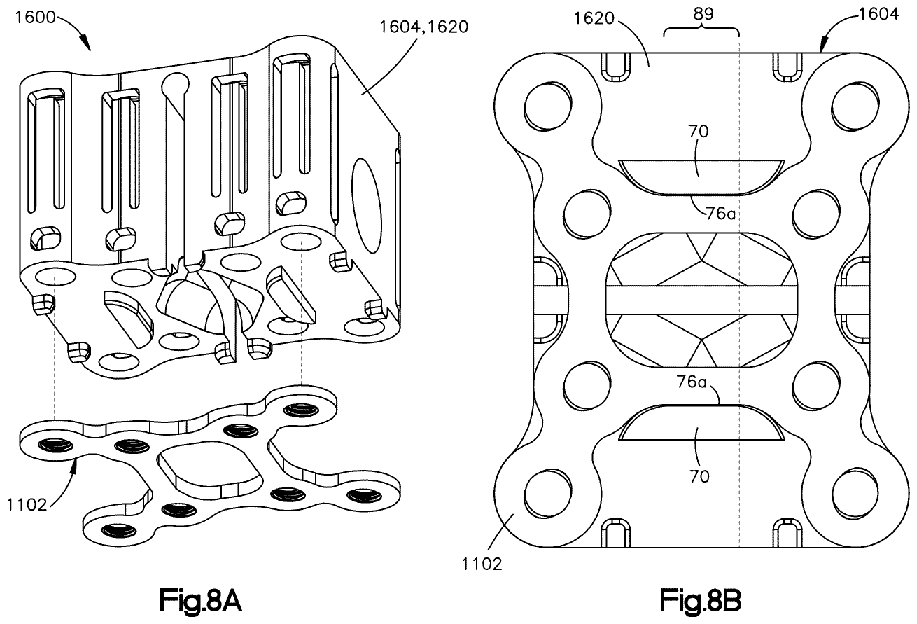

Referring now to FIGS. 8A-8B, in additional embodiments, a bone plate system 1600 can employ a guide 1604 configured for use with the large X-plate 1102 described above with reference to FIG. 6A. In the present example embodiment, the guide 1604 is a “wide” or “large” version of the X-guide 1504 described above with reference to FIGS. 7A-7D. Thus, the guide 1604 can be referred to as a “wide” or “large X-guide” 1604. The large X-guide 1604 can be substantially similar to the X-guide 1504 described above, but having the guide body 1620 lengthened along the second direction X. As above, in the presently illustrated example, the portion of the guide body 1620 that is lengthened along the second direction X is the portion 89 located between the lateral extremities of the first surface portions 76a of the first pair of engagement members 70.

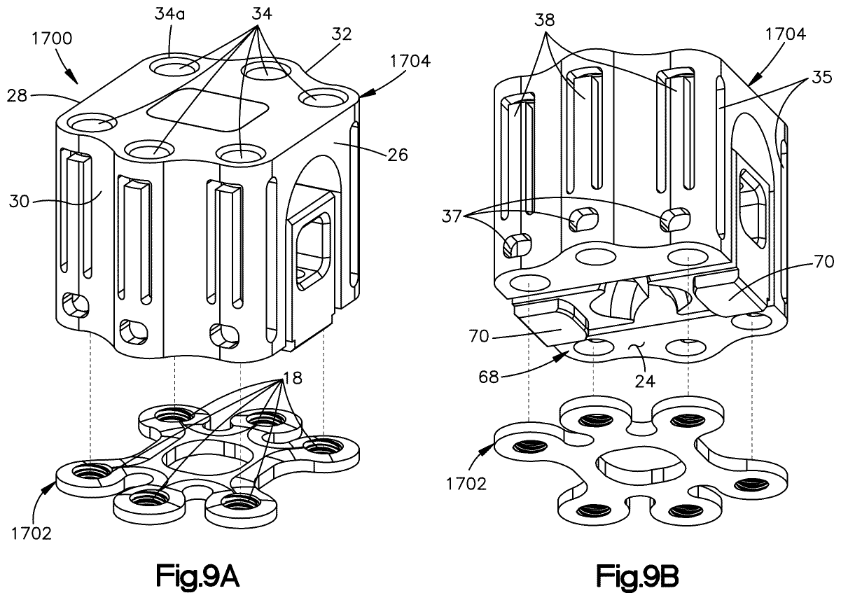

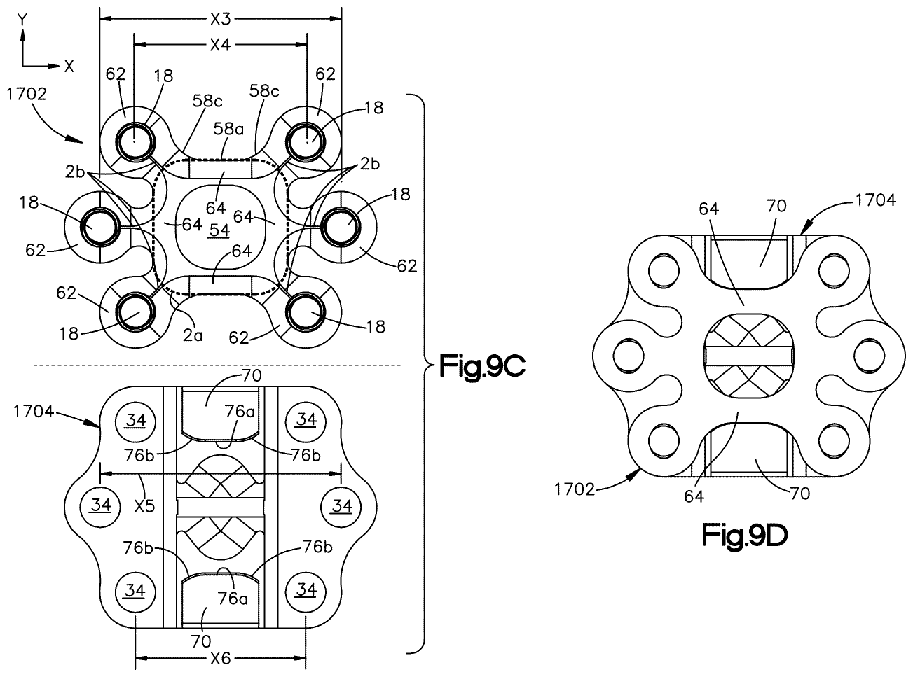

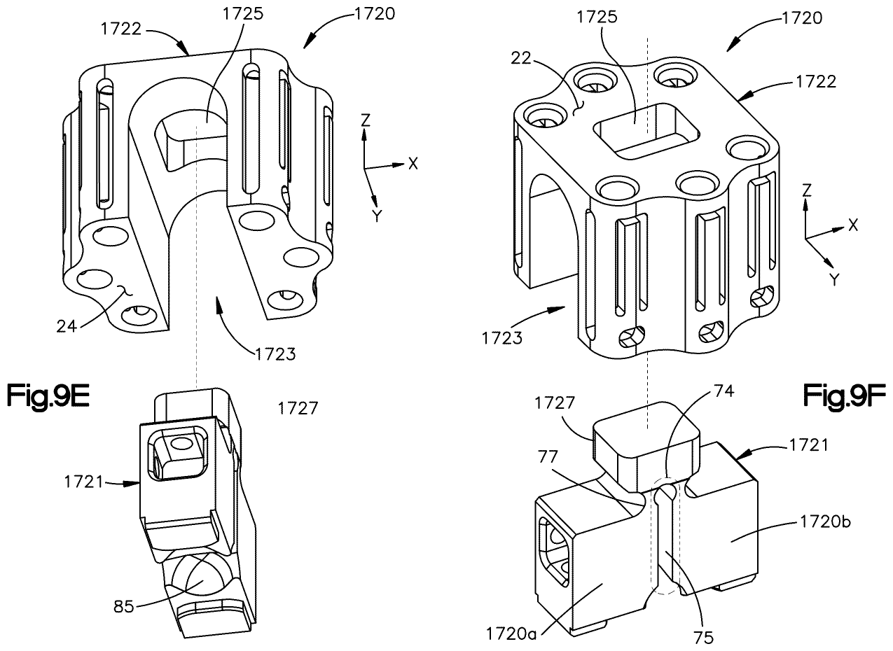

Referring now to FIGS. 9A-9F, a bone plate system 1700 can employ a bone plate 1702 having an exemplary “star plate” configuration and an associated guide 1704, which can be referred to as a “star guide” 1704. As shown in FIG. 9C, the star plate 1702 has six (6) arms 2b extending from a central hub portion 2a. The hub portion 2a of the star plate 1702 has four (4) bridges 64 arranged in a rectangular pattern, with a first pair of the bridges 64 opposite each other along the second direction X and a second pair of the bridges 64 opposite each other along the third direction Y. As with embodiments described above, the star plate 1702 has an aperture 54, which can be centrally located within the hub portion 2a. In the illustrated example, two (2) of the arms 2b extend outwardly from the first pair of bridges 64 (i.e., the pair opposite each other along the second direction X), preferably at a midpoint of the star plate 1702 along the third direction Y. These two (2) arms 2b can be referred to as the “middle arms” 2b. Four (4) of the arms 2b extend outwardly from four (4) corners of the hub portion 2a (i.e., where the bridges 64 intersect each other) at oblique angles relative to both the second and third directions X, Y. These four (4) arms 2b can be referred to as the “corner arms” 2b. As shown, the middle arms 2b can provide a hole spacing X3 along the second direction X that is greater than the hole spacings X4 of the corner arms 2b along the second direction X. In the illustrated example, the second pair of bridges 64 (i.e., the pair opposite each other along the third direction Y) have exterior bridge side surfaces 58a configured to be engaged and clamped by the star guide 1704, as described in more detail below.

The star guide 1704 has a guide body 1720 that defines a plurality of channels 34, which preferably correspond to and align with each of the holes 18 of the star plate 1702 when the star guide 1504 and star plate 1702 are coupled together in a preferred orientation relative to each other, such as the orientation shown in FIGS. 9A-9B and 9D. In particular, the guide body 1720 defines four (4) corner channels 34 that align with the four (3) corner plate holes 18 and also defines two (2) middle channels 34 that align with the two (2) middle plate holes 18. Accordingly, the two (2) middle channels 34 have a channel spacing distance X5 that is substantially equivalent to the hole spacing distance X3 of the middle plate holes 18 and the four (4) corner channels 34 have a channel spacing distance X6 that is substantially equivalent to the hole spacing distance X4 of the corner plate holes 18 of the star plate 1702. The guide body 1720 has a proximal or top surface 22 and a distal or bottom surface 24 opposite each other along the first direction Z. The guide body 1720 has a first side 26 and a second side 28 opposite each other along the third direction Y and a third side 30 and a fourth side 32 opposite each other along the second direction Y. To provide the channels 34 with a complimentary arrangement with the plate holes 18, the third and fourth sides 30, 32 of the guide body 1720 preferably flare outwardly along a middle portion of the guide body 1720. The channels 34 can otherwise be configured similar to the channels 34 of the embodiments described above. Accordingly, the channels 34 can extend from an upper channel perimeter 34a at a boundary with the top surface 22 of the guide body 1720, and each upper channel perimeter 34a preferably extends an entire revolution about the respective central channel axis Z2 in contiguous fashion with the top surface 22. Additionally, the guide body 1720 includes fastener retention mechanism 38 associated with each channel 34 and configured similar to those described above.

The coupling mechanism 68 includes a pair of engagement members 70, which can be configured similar to the engagement members 70 described above. The engagement members 70 of the present embodiment are configured to engage and clamp against the exterior bridge side surfaces 58a of the second pair of bridges 64, as shown in FIG. 9C. The guide body 1720 can also define a decoupling structure, such as a recess 85, which can be configured similar to the recess 85 described above with reference to FIGS. 3B-3C.

In the illustrated embodiment, the guide body 1720 includes a first body member 1721 that has a coupling mechanism 68 for coupling with the star plate 1702 and a second body member 1722 that defines the channels 34. As shown in FIGS. 9C-9D, the first and second body members 1721, 1722 can be separate members that are attachable with each other, which can facilitate ease of manufacturability of the guide body 1720. In the illustrated embodiment, the second body member 1722 defines a receptacle 1723 configured to receive an associated portion of the first body member 1721. The receptacle 1723 extends upwardly along the first direction Z from a bottom surface 24 of the second body member 1722 towards a top surface 22 thereof. The second body member 1722 preferably also has a receiving structure 1725 for mounting with a complimentary mounting formation 1727 of the first body member 1721. As shown, the receiving structure 1725 can be a receiving aperture 1725 defined by the second body member 1722 and extending downwardly from the top surface 22 to the receptacle 1723. The mounting formation 1727 of the first body member 1721 can be a mounting block 1727 having a complimentary geometry with that of the receiving aperture 1725, such that when the mounting block 1727 is received within the receiving aperture 1725, their complimentary geometries retain the relative positions between the first and second body members 1721, 1722 with respect to the second and third directions X, Y. It should be appreciated that, in other embodiments, the first and second body members 1721, 1722 can be monolithic with each other.

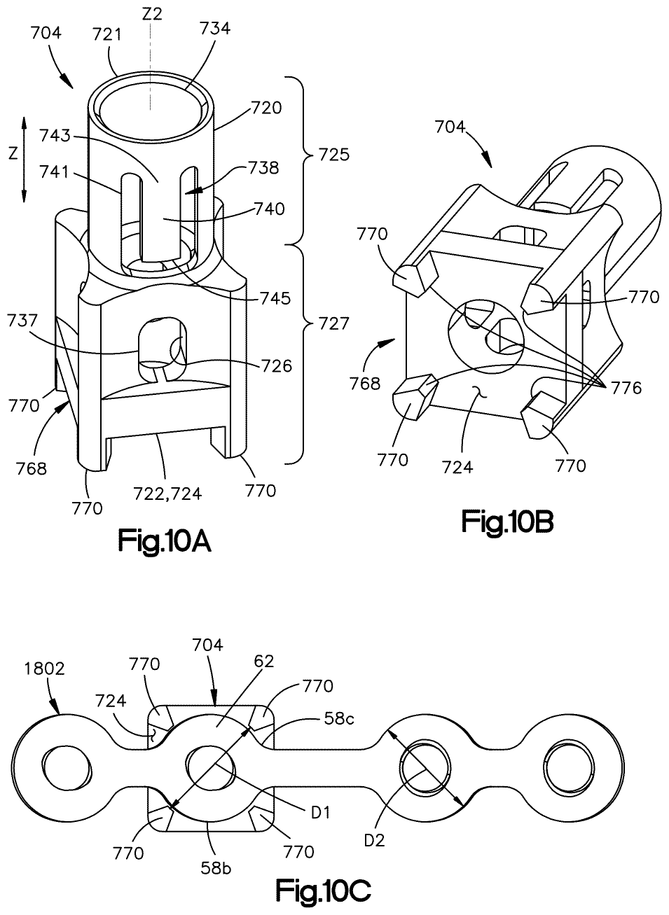

Referring now to FIGS. 10A-10C, a single-channel screw guide 704 can be employed with a bone plate system for coupling with a single hole 18 of a bone plate, such as the straight plate 1802 illustrated in FIG. 10C, and any of the other types of bone plates described herein, including bone plates 2, 102, 202, 302, 402, 1002, 1102, 1202, 1402, 1702, by way of non-limiting examples. The single-channel guide 704 has a guide body 720 having a proximal end 721 and a distal end 722 spaced from each other along the first direction Z. The guide body 720 defines a channel 734 that extends from the proximal end 721 to a bottom surface 724 at the distal end 722 along a central channel axis Z2. The guide body 720 can also have a proximal handle portion 725 and a distal mounting portion 727 spaced from each other along the first direction Z. As shown in FIG. 10A, the proximal handle portion 725 can have a cylindrical geometry. The distal mounting portion 727 can have a general rectangular geometry, which can facilitate quick visual indexing of the position of engagement members 770 of the guide body 720, which engagement members 770 are discussed in more detail below. The guide body 720 preferably defines one or more visualization windows 737 that extend through a sidewall 726 of the guide body 720 and are open to the channel 734. In the illustrated embodiment, the guide body 720 defines four (4) visualization windows spaced at 90-degree intervals about the central channel axis Z2, although fewer or more than four (4) visualization windows 737 are within the scope of the present embodiment.

The guide body 720 can also include a screw retention mechanism 738, which can be similar to those described above. For example, the screw retention mechanism 738 can include at least one compliant member 740 that extends within the channel 734 and is configured to contact the screw 6 and apply a retention force thereto sufficient to retain the screw within the channel 734 in the pre-loaded position at least against the force of gravity. As above, the screw retention mechanism 738 is also preferably configured such that the retention force is also sufficient to hold the screw 6 in the pre-loaded position while the surgeon couples the driver to the drive socket of the screw head 44 and subsequently allowing the screw 6 to decouple from the screw retention mechanism 38 responsive to a threshold driving force applied to the screw 6 along the screw axis Z3. The compliant member 740 can be a flexible tab 740 defined by a cutout portion 741 of the guide body 720. The cutout portion 741 can have a U-shape and the flexible tab 740 can extend downwardly from a first, connected tab end 743 to a second, free tab end 745. In other embodiments, the flexible tabs 740 can extend upwardly (toward the proximal end 721) from the connected tab end 743 to the free tab end 745. Other flexible tab 740 configurations are also within the scope of the present disclosure. It should be appreciated that the cutout portion 741 can effectively define an additional visualization window for viewing the bone screw 6 within the channel 734. As shown, the screw retention mechanism 738 can be defined by the proximal handle portion 725 of the guide body 720.

The distal mounting portion 727 of the guide body 720 includes a coupling mechanism 768 that includes a plurality of engagement members 770 that extend downward from the bottom surface 724 along the first direction Z. As shown in FIG. 10B, the plurality of engagement members 770 can include four (4) engagement members 770 spaced from each other in a rectangular (e.g., square) pattern around and radially spaced from the channel 734. The engagement members 770 are configured to extend alongside respective side surface portions of the plate body 1812 of the bone plate 1802 in a manner maintaining the angular orientation of the guide body 720 within a limited angular range of rotation relative to the bone plate 1802 about the central hole axis Z1. The engagement members 770 each have an interior surface 776 configured to face and engage with an exterior side surface 58 of the bone plate. In the illustrated embodiment, the interior surfaces 776 are configured to interface with exterior node side surfaces 58b. Preferably, the single-channel guide 704 is configured to couple with portions of the bone plate 1802 even if those portions have been bent or otherwise contoured to the underlying bone. As shown in FIG. 10C, the engagement members 770 are spaced from each other such that a distance D1 between opposed interior surfaces 776 is slightly greater than a diameter D2 of the exterior node side surfaces 58b, thereby providing a measure of tolerance for allowing the coupling mechanism 768 to couple with nodes 62 or other portions of the bone plate 1802 that have been bent or otherwise contoured to the underlying bone. The interior surfaces 776 are also configured to interface with the exterior side relief surfaces 58c of the bone plate 1802 in a manner that provides the guide 704 with a measure of toggling (i.e., back and forth) rotation about the central hole axis Z1 when coupled to a node, but limits or otherwise bounds the angle of rotation within a limited angular range of rotation.

With reference to FIGS. 11-13B, additional example embodiments of screw retention mechanisms will now be described.

Referring now to FIG. 11, a guide 104 can have a guide body 120 having a screw retention mechanism 138 in which a compliant member 140 thereof can be a flexible tab 140 that extends downwardly from a connected tab end 143 to a free tab end 145. Accordingly, in this embodiment, the flexible tab 140 can be defined by a U-shaped cutout portion 141 of the guide body 120. In this embodiment, the screw retention mechanism 138 can be located adjacent a proximal end 121 of the guide body 120 and remote from a distal end 123 of the guide body 120. It should be appreciated that the guide body 120 of the present embodiment can otherwise be generally similar to the guide body 20 described above and other guide bodies described herein.

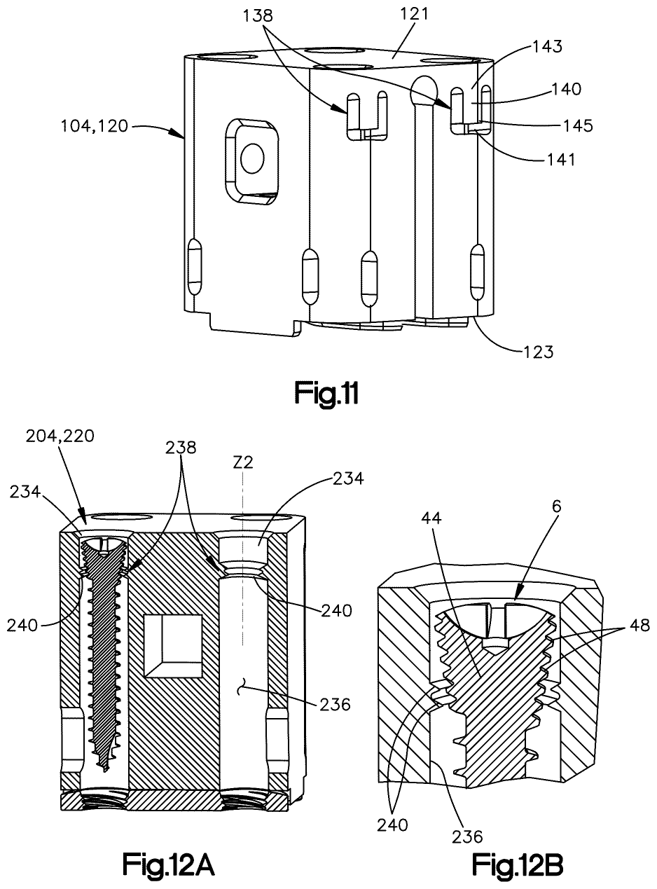

Referring now to FIGS. 12A-12B, in another embodiment, a guide 204 can have a guide body 220 having a screw retention mechanism 238 that includes internal threads 240 located within channel(s) 234 of the guide body 220. In such embodiments, the internal threads 240 can be configured to engage at least some of the exterior threads 48 on the head 44 of a bone screw 6 pre-loaded within the channel 234 to apply the retention force to the screw head 44. The internal threads 240 preferably extend radially inwardly from an interior surface 236 of the channel 234 toward the central channel axis Z2. The internal threads 240 have a minor thread diameter that is slightly less than a major thread diameter of at least some of the exterior threads 48 on the head 44, thereby at least preventing the screw 6 from dropping through the channel 34 responsive to gravity. Similar to the embodiment described above, the retention force applied by the internal threads 240 can also be sufficient to hold the screw 6 in the pre-loaded position while the surgeon couples the driver to the drive socket of the screw head 44. As shown, the internal threads 240 can be defined by the guide body 220, although in other embodiments the internal threads 240 can be defined by a separate piece, such as a ring-member or collar-member, positioned within the channel 234. It should also be appreciated that the internal threads 240 can be adapted as needed to provide different retention force magnitudes.

In yet other embodiments, the screw retention mechanism 238 can include an annular protrusion that extends radially inwardly from the interior surface 236 of the channel 234 and is substantially devoid of threads in a first configuration. In such embodiments, the annular protrusion can be configured such that the external threads 48 of the screw head 44 deform, cut, tap, or otherwise form the internal threads 240 in the annular protrusion during the screw pre-loading process, during which the engagement between the external threads 48 of the screw head 44 and the annular protrusion provides the retention force.

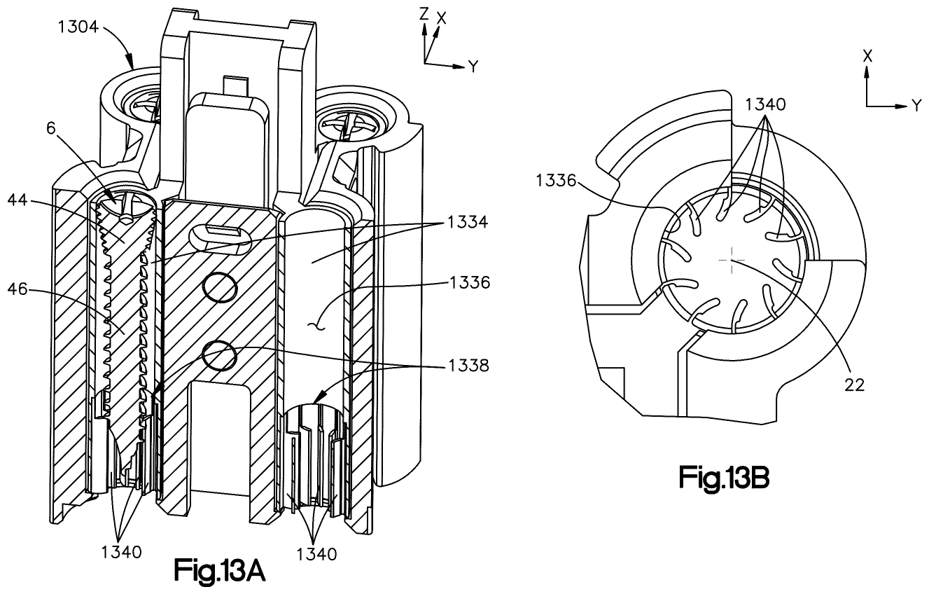

Referring now to FIGS. 13A-13B, in yet another embodiment, a retention mechanism 1338 for retaining a bone screw 6 within a channel 1334 of a guide 1304 can include a plurality of fingers 1340 extending radially inward from an interior surface 1336 of the channel 1334. As shown, the fingers 1340 can be located along a distal portion of the channel 1334 and can be configured to engage (and apply the retention force to) the screw shaft 46. Alternatively, the fingers 1340 can be located along a select portion of the channel 1334 for engaging the screw head 44. As shown, the fingers 1340 can have curved profiles in the X-Y plane. Preferably, in such embodiments, the fingers 1340 curve toward a rotational direction associated with the respective rotational screw direction the causes the screw 6 to advance through the underlying bone 1. It should be appreciated that the fingers 1340 can have other shapes and configurations for applying the retention force to the screw 6.

It should be appreciated that the foregoing screw retention mechanisms are provided as non-limiting examples for retaining the screw(s) 6 within the channel(s) 34, 134, 234, 1334 and that various other retention mechanism types and designs can be employed.

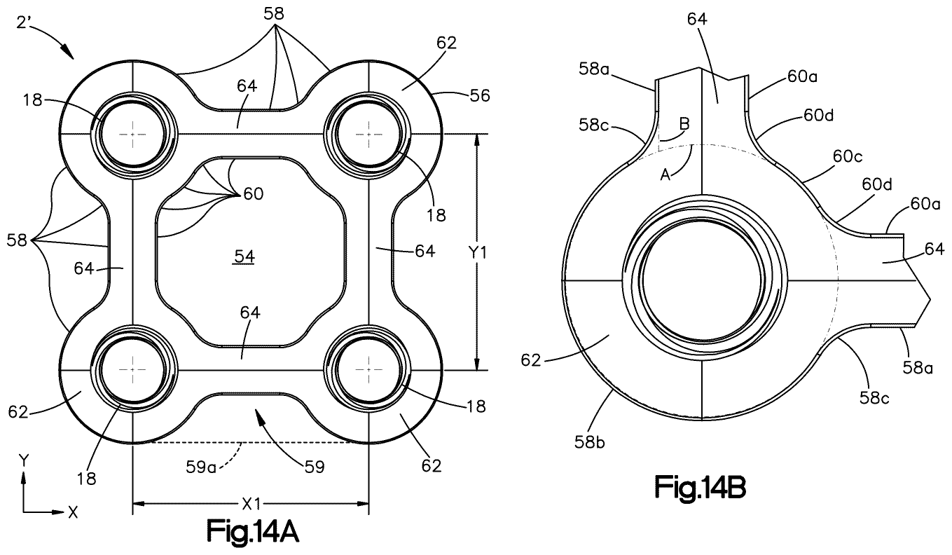

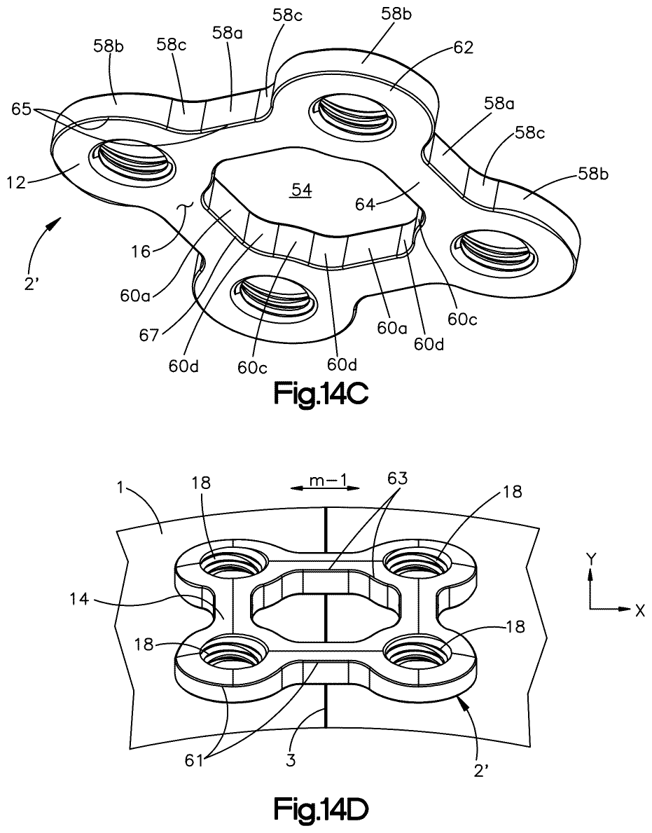

With reference to FIGS. 14A-20C, additional example embodiments of bone plating assemblies will now be described that include various screw guides 4′, 304, 404, 504, 604, 804 and various bone plates 2′, 102′, 202′, 802, 502, 602, 702, 802, 1702′. The various bone plates shown in FIGS. 14A-20C have a hub structure similar to those of bone plates 2, 102, 202, 302, 402, 1002, 1102, 1202, 1402, described above; accordingly, the guides 4′, 304, 404, 504, 604, 804 in these embodiments have respective coupling mechanisms configured to couple with the hub structure. For the sake of brevity, the following discussion focuses on some of the differences employed in the bone plates 2′, 102′, 202′, 802, 502, 602, 702, 802, 1702′ and screw guides 4′, 304, 404, 504, 604, 804 of FIGS. 14A-20C relative to the bone plates and screw guides described above with reference to FIGS. 1A-9F. Furthermore, in the following discussion, features of the bone plates and screw guides that have similar design and function to those described above can employ the same reference numbers.

Referring now to FIGS. 14A-14D, another embodiment of a bone plate 2′ has a geometry similar to that of the bone plate 2 described above with reference to, e.g., FIGS. 2A-2C, but can include different geometries at portions thereof, such as along the interior side surfaces 60 located within the aperture 54. For example, in the present embodiment, the interior side surfaces 60 (within the aperture 54) can also include interior node side surfaces 60c, as best shown in FIG. 14B. Additionally, adjacent each node 62 of the bone plate 2′, the interior side surfaces 60 can also include a pair of interior side relief surfaces 60d extending oppositely away from the interior node side surface 60c to respective adjacent interior bridge side surfaces 60a. In this manner, the interior side relief surfaces 60d are located at intersections between the nodes 62 and bridges 64 within the aperture 54. The interior side relief surfaces 60d can beneficially reduce stress concentrations in the plate body 12. Additionally, the plate body 12 of bone plate 2′ can define at least one upper chamfer surface 61 between the outer surface 14 and the exterior side surfaces 58 of the plate body 12 and also at least one chamfer surface 63 between the outer surface 14 and the interior side surfaces 60 of the plate body 12 (see FIG. 14D). The plate body 12 of bone plate 2′ can also define at least one chamfer surface 65 between the bone-facing surface 16 and the exterior side surfaces 58 of the plate body 12 and also at least one chamfer surface 67 between the bone-facing surface 16 and the interior side surfaces 60 of the plate body 12 (see FIG. 14C).

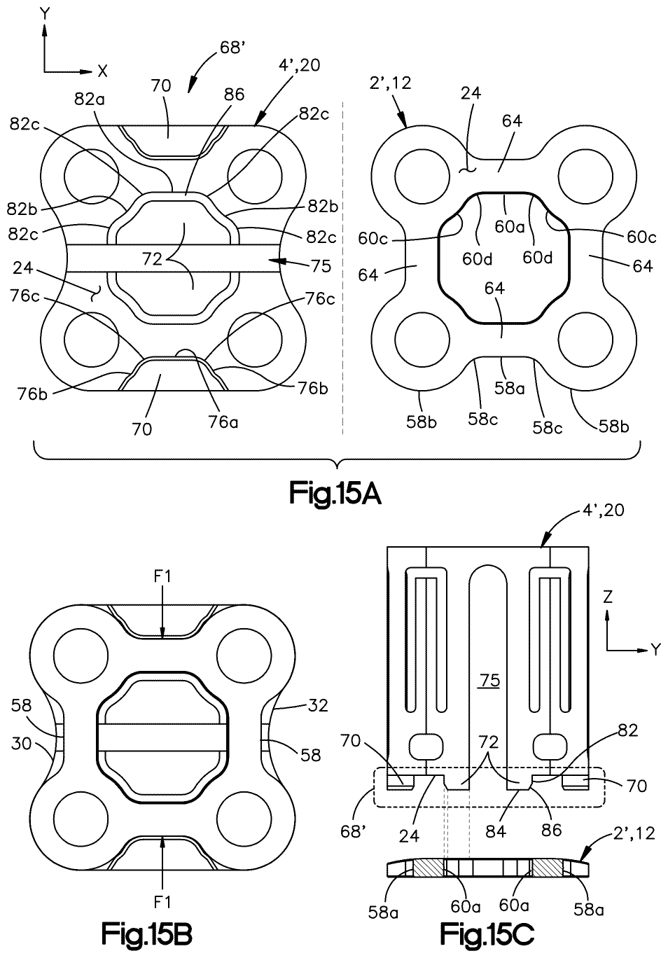

Referring now to FIGS. 15A-15B, in an additional embodiment, a fastener guide 4′ has a coupling mechanism 68′ that can be configured to engage with the interior side surfaces 60 of the bone plate 2′. In the present embodiment, the coupling mechanism 68′ includes a plurality of engagement members 70, 72 that includes a first pair of engagement members 70 and a second pair of engagement members 72 that each protrude downwardly from the bottom surface 24 of the plate body 12 along the first direction Z. The first pair of engagement members 70 of the present embodiment are similar to the engagement members 70 described above with reference to FIGS. 3A-3G, and are thus configured to clamp against the exterior side surfaces 58 of the plate body 12 with inward bias force F1 along the third direction Y (FIG. 15B). The second pair of engagement members 72 are spaced inwardly from the first pair of engagement members 70 along the third direction Y. The second pair of engagement members 72 are configured to extend within the coupling aperture 54 and engage one or more of the interior side surfaces 60 of the plate body 12 therein for centering the guide 4′ in the desired orientation relative to the bone plate 2′, particularly such that the channels 34 of the guide 4′ are concentrically aligned with the plate holes 18. Similar to the first pair of engagement members 70, one of the second pair of engagement members 72 is located on one side of the slot 75 and the other engagement member 72 is located on the other side of the slot 75.

As in embodiments described above, the interior surfaces 76 of the first pair of engagement members 70 each include a first surface portion 76a configured to engage one of the exterior bridge side surfaces 58a and also one or more second surface portions 76b configured to engage one or more of the exterior relief surfaces 58c and/or exterior node side surfaces 58b. However, in the present embodiment, as shown in FIG. 15A, the interior surfaces 76 can further include one or more third surface portions 76c configured to engage one or more of the exterior side relief surfaces 58c.

The second pair of engagement members 72 in the present embodiment each have an exterior surface 82 that extends downward from the bottom surface 24 of the guide body 20 toward a bottom surface 84 of the engagement member 72. The second pair of engagement members 72 can each also include at least one chamfer surface 86 extending between the exterior surfaces 82 and the bottom surfaces 84. The exterior surfaces 82 of the second pair of engagement members 72 are configured to engage associated interior side surfaces 60 of the plate body 12 in a manner that centers the guide 4 in the desired orientation relative to the bone plate 2′. As shown in FIG. 15A, each exterior surface 82 of the present embodiment includes a first surface portion 82a configured to engage one of the interior bridge side surfaces 60a. The exterior surfaces 82 of the present embodiment can also include one or more second surface portions 82b configured to engage one or more of the interior node side surfaces 60b and can further include one or more third surface portions 82c configured to engage one or more of the interior side relief surfaces 60c. Such engagement between the surface portions 82a-c of the second pair of engagement members 72 and the interior side surfaces 60 of the bone plate 2′ provides a centering function that helps maintain alignment of the guide 4′ at the desired orientation relative to the bone plate 2′ (i.e., so that the plate holes 18 are aligned with the channels 34 of the guide 4), as shown in FIG. 15B. Additionally, as in the embodiments above, engagement of the second and third surface portions 76b,c of the first pair of engagement members 70 against the exterior node and relief side surfaces 58b,c can also provides a supplemental centering function.

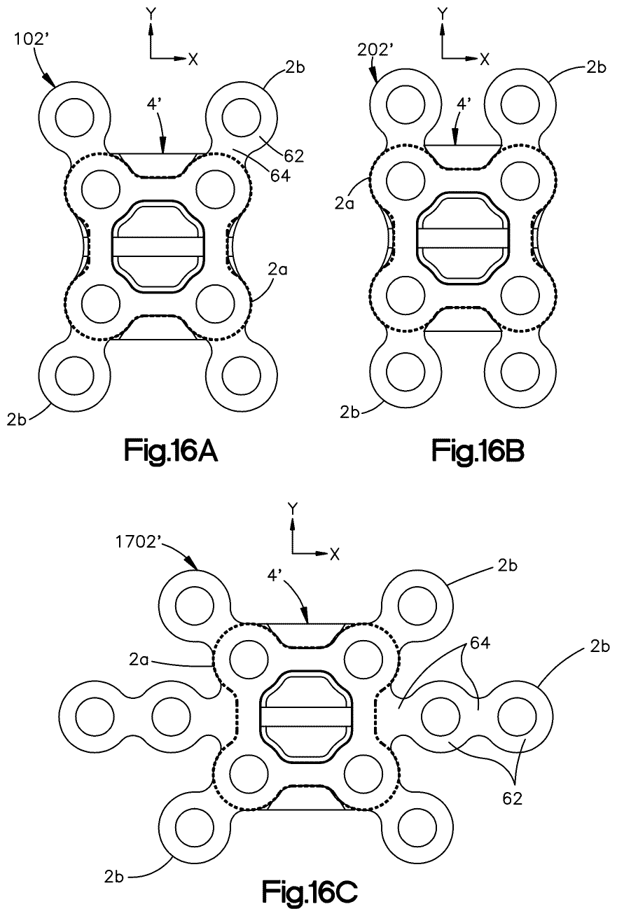

Referring now to FIGS. 16A-16C, additional example bone plates 102′, 202′, 1702′ are shown that employ the design of the bone plate 2′ described above with reference to FIGS. 14A-15C as a hub or bracket portion 2a from which additional plate portions, such as plate arms 2b, can extend. Similar to the extended-area bone plates described above, such as with reference to FIGS. 4A-4D, the additional plate portions (e.g., arms 2b) shown in FIGS. 16A-16C increase the plate coverage area for underlying bone and provide additional plate holes 18. FIG. 16A shows an example “X-plate” style bone plate 102′, similar to the bone plate 102 shown in FIG. 4A. FIG. 16B shows an example “H-plate” style bone plate 202′, similar to the bone plate 202 shown in FIG. 4B. FIG. 16C shows an example “star plate” style bone plate 1702′ that is generally similar to the star plate 1702 shown in FIGS. 9A-9D and has six (6) arms 2b extending from the hub portion 2a. In the present embodiment, the star plate 1702′, the four (4) corner arms 2b extend outwardly from the four (4) nodes 62 of the hub portion 2a at oblique angles relative to both the second and third directions X, Y. The two (2) middle arms 2b extend outwardly from the pair of bridges 64 that are opposite each other along the second direction X), preferably at a midpoint of the star plate 1702′ along the third direction Y. Additional sternal bone plate types and designs can employ the design of the bone plate 2′ as a hub portion 2a, including T-plates, double-T plates, double-H plates, L-plates, angled plates, and straight plates (for affixation to various ribs), by way of non-limiting examples. It should also be appreciated that the hub portion 2a design of the bone plate 2′ can be employed on numerous types, styles, and sizes of bone plates for indications other than sternal fixation.

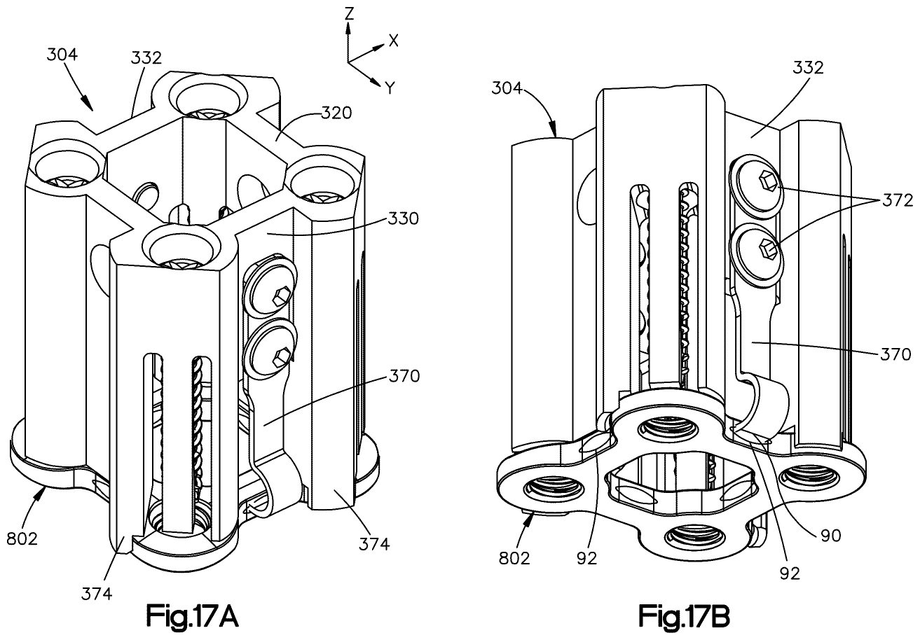

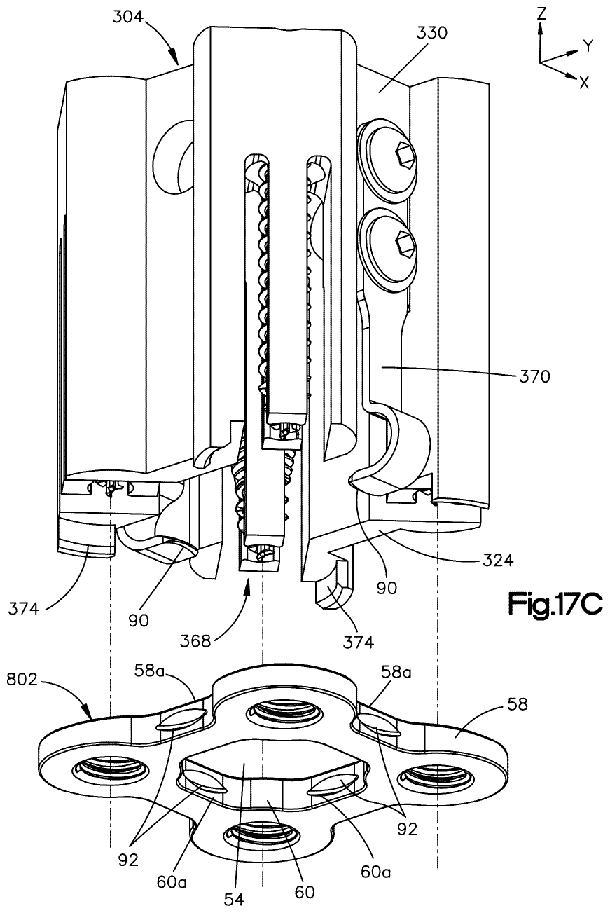

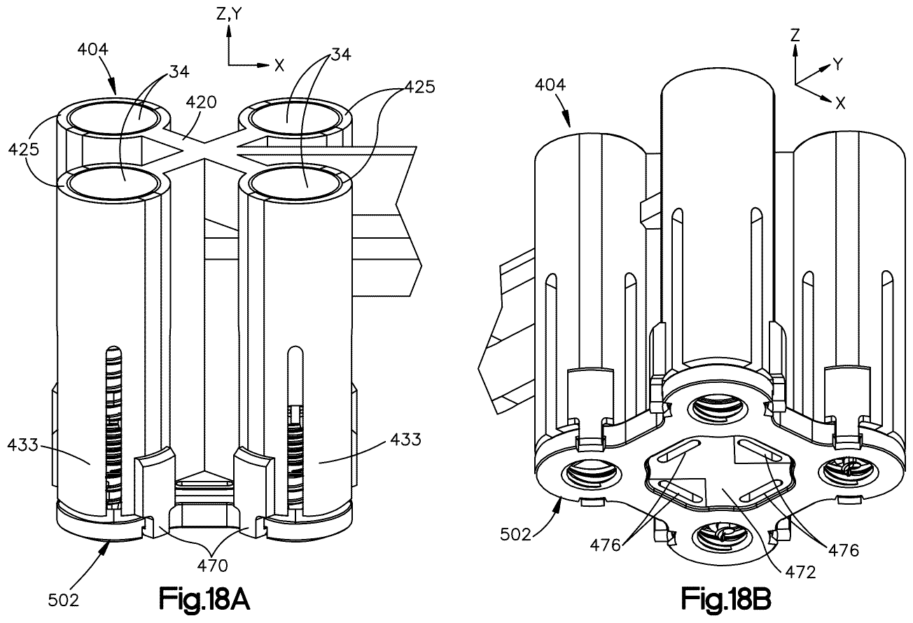

With reference to FIGS. 17A-20C, additional example embodiments of screw guides 304, 404, 504, 604 are shown that have additional and/or alternative features than those of the various screw guides described above. For example, the following screw guides 304, 404, 504, 604 described with reference to FIGS. 17A-20C have various coupling mechanisms that employ protrusions 90 that extend from the respective guide body and extend within recesses 92 defined in various side surfaces 58, 60 of the respective bone plate 402, 502. Such protrusion-recess 90, 92 engagement between the guides and the associated bone plates can increase the coupling retention force between the guide and the bone plates. For the sake of brevity, the following discussion focuses on some of the differences employed in the screw guides 304, 404, 504, 604 and bone plates 402, 502 of FIGS. 17A-20C relative to the various screw guides and bone plates described above with reference to FIGS. 1A-16C. In the following discussion, features having similar design and function to those described above can employ the same reference numbers.

Referring now to FIGS. 17A-17C, an exemplary screw guide 304 is configured to couple with a bone plate 802 having recesses 92 defined within exterior side surfaces 58 of the plate 802. The bone plate 802 is similar to the bone plate 2′ described above with reference to FIGS. 14A-14D but includes recesses 92 defined within the exterior side surfaces 58, particularly within the exterior bridge side surfaces 58a. In the illustrated example, the bone plate 802 also includes recesses 92 defined within interior side surfaces 60 in the aperture 54, particularly in the interior bridge side surfaces 60a, although these interior recesses 92 can be omitted in the present embodiment. The screw guide 304 has a coupling mechanism 368 that includes a pair of spring arms 370 extending downward from opposed sides 330, 332 of the guide body 320. The spring arms 370 have distal ends that include the protrusions 90, which face toward each other and are configured to extend within the recesses 92 in the exterior bridge side surfaces 58a. The spring arms 370 are flexible to facilitate easy coupling and decoupling with the bone plate 802. The spring arms 370 can also be attachable to the sides 330, 332 of the guide body 320 via fasteners 372. The screw guide 304 can also include one or more support tabs 374 that extend below the bottom surface 324 of the guide body 320 and extend alongside exterior side surfaces 58 of the bone plate 802.

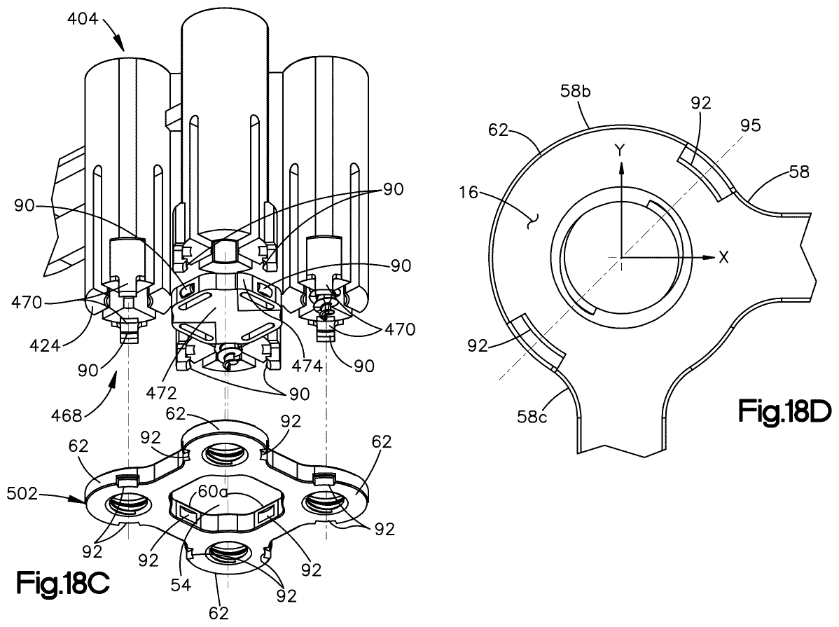

Referring now to FIGS. 18A-18D, an exemplary screw guide 404 is configured to couple with a bone plate 502 having recesses 92 defined within exterior side surfaces 58 of the plate 502. The bone plate 502 of the present example is similar to the bone plates 2′, 802 described above but includes recesses 92 defined within the exterior node side surfaces 58b. In particular, each node 62 in this example includes a pair of recesses 92 opposite each other along an axis 95 that is angularly offset at substantially 45 degrees from both the second and third directions X, Y, as shown in FIG. 18D. The recesses 92 on the nodes 62 can also extend into the exterior relief side surfaces 58c. The bone plate 502 also includes recesses 92 defined in the interior side surfaces 58 within the aperture 54, particularly in the interior bridge side surfaces 62a.

The screw guide 404 has a guide body 420 and a coupling mechanism 468 that includes a plurality of latch arms 470 that extend downwardly from a bottom surface 424 of the guide body 420. In the illustrated embodiment, the guide body 420 has individual tube portions 425 in which the channels 34 are defined, with each tube portion 425 having a pair of flexible latch arms 470 that extend downwardly from outer surfaces 433 of the tube portions 425. Each pair of latch arms 470 have respective protrusions 90 that extend toward each other and are configured to extend within respective recesses 92 in the exterior node side surfaces 58b of the bone plate 502.

The coupling mechanism 468 also includes a hub member 472 that extends below the bottom surface 424 and has an exterior surface 474, which preferably has a complimentary geometry with the interior side surfaces 58 of the bone plate 502 within the aperture 54. The exterior surface 474 of the hub member 472 has additional protrusions 90 that extend therefrom and are configured to extend within the recesses 92 in the interior bridge side surfaces 60a of the bone plate 502. The hub member 472 can include spring members, such as spring relief slots 476, that extend inwardly of the hub protrusions 90 and provide the hub protrusions 90 with flexibility to extend within and decouple from the associated recesses 92 of the bone plate 502. Accordingly, the hub member 472 can provide additional coupling force between the guide 404 and the bone plate 502. In other embodiments, the guide 404 can omit the hub member 474 and rely upon the flexible latch arms 470 for coupling with the bone plate 502. Alternatively, the guide 404 can omit the flexible latch arms 470 are rely upon the hub member 474 for coupling with the bone plate 502.

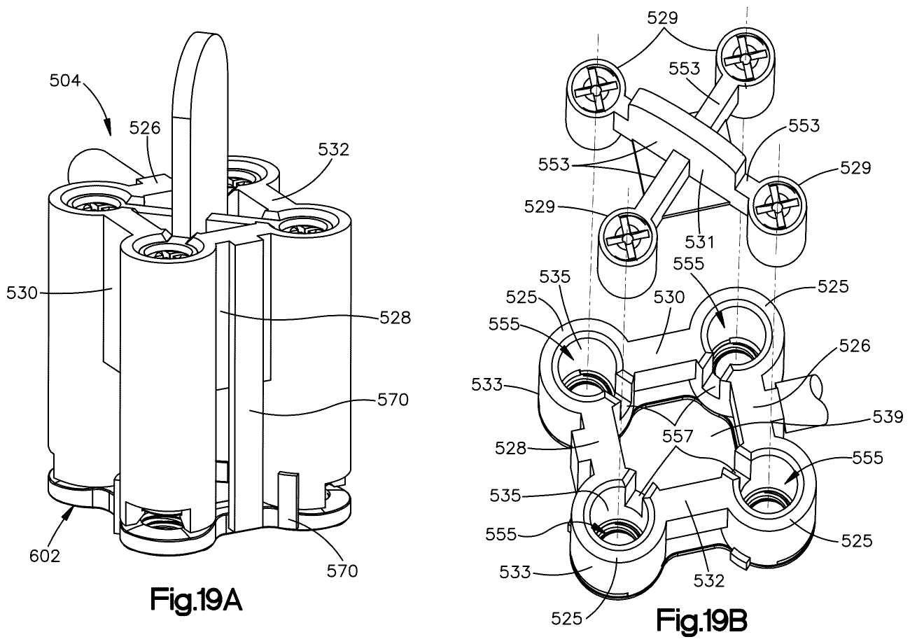

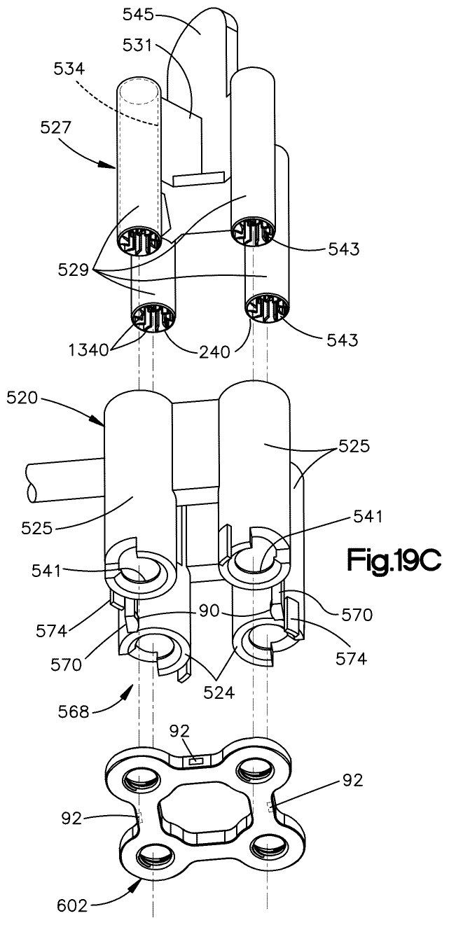

Referring now to FIGS. 19A-19C, an exemplary screw guide 504 is configured to couple with a bone plate 602 having recesses 92 defined within exterior side surfaces 58 of the plate 602. In the illustrated embodiment, the guide 504 has an outer housing body 520 (also referred to as the “outer housing” 520) and an inner housing body or “cartridge” 527 that carries the bone screws 6 and is insertable within an interior space 539 of the outer housing 520, as described in more detail below.

The bone plate 602 is similar to the bone plates 2′, 802, 502 described and includes recesses 92 defined within the exterior side surfaces 58, particularly within the exterior bridge side surfaces 58a. The guide 504 has a coupling mechanism 568 that includes a pair of flexible arms 570 extending downward from opposed sidewalls 526, 528 of the outer housing 520. The flexible arms 570 have distal ends that include respective protrusions 90, which face toward each other and are configured to extend within the recesses 92 in the exterior bridge side surfaces 58a. The spring arms 570 are flexible to facilitate easy coupling and decoupling with the bone plate 602. The screw guide 504 can also include one or more support tabs 574 that extend below the bottom surface 524 of the outer housing 520 and extend alongside exterior side surfaces 58 of the bone plate 602.

The outer housing 520 defines tubular portions 525 having outer tube surfaces 533 and interior tube surfaces 535 opposite each other. The interior tube surfaces 535 define barrel receptacles 555. The outer housing 520 also defines the opposed sidewalls 526, 528, that are spaced from each other along the second direction X, and opposed sidewalls 530, 532 spaced from each other along the third direction Y. The interior space 539 of the outer housing 520 is defined between interior surfaces of the sidewalls 526, 528, 530, 532.

The cartridge 527 has barrel portions 529 that define the channels 534 therein. The cartridge 527 includes a bracket portion 531 having bracket arms 553 that interconnect the barrel portions 529. The cartridge 527 can include an optional handle tab 545 for inserting and withdrawing the cartridge 527 within the housing body 520 as needed. The bracket portion 531 is configured for insertion within the interior space 539 and the barrel portions 529 are configured to reside within the barrel receptacles of the tubular portions 525. The tubular portions 525 can also define slots 557 extending between the outer and interior tube surfaces 533, 535 for receiving the bracket arms 553 of the cartridge 527. The outer housing 520 defines one or more landing surfaces 541 upon which mating surfaces of the cartridge 527 sit when the cartridge 527 is fully seated within the housing body 520. In the illustrated embodiment, the landing surfaces 541 are located within the tubular members 525 at bottom ends of the interior tube surfaces 535, such that bottom ends 543 of the barrel portions 529 of the cartridge 527 abut the landing surfaces 541 when the cartridge 527 is fully seated. As shown in FIG. 19C, the cartridge 527 can include a screw retention mechanism in each channel 534, such as fingers 1340 similar to those described above with reference to FIGS. 13A-13B. It should be appreciated that the cartridge 527 can employ other types of retention mechanisms, including the other types described above.

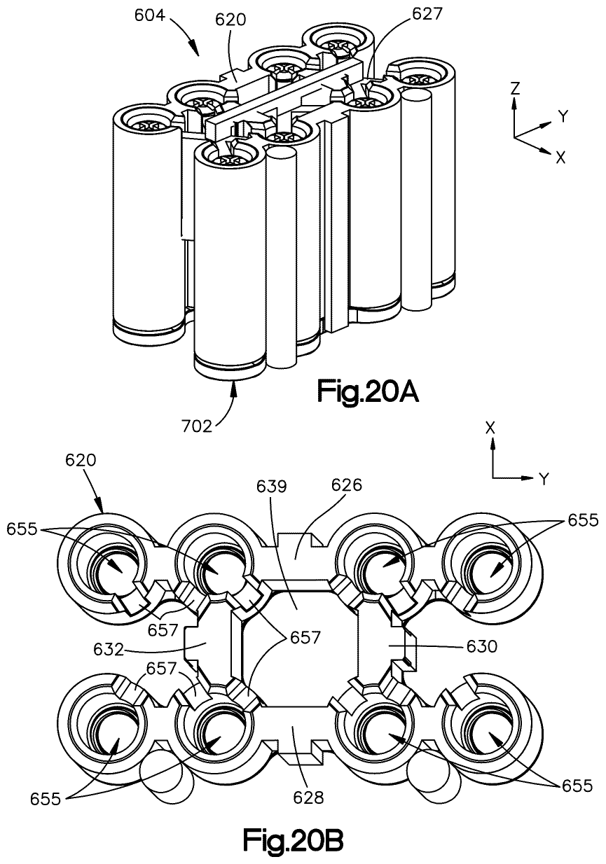

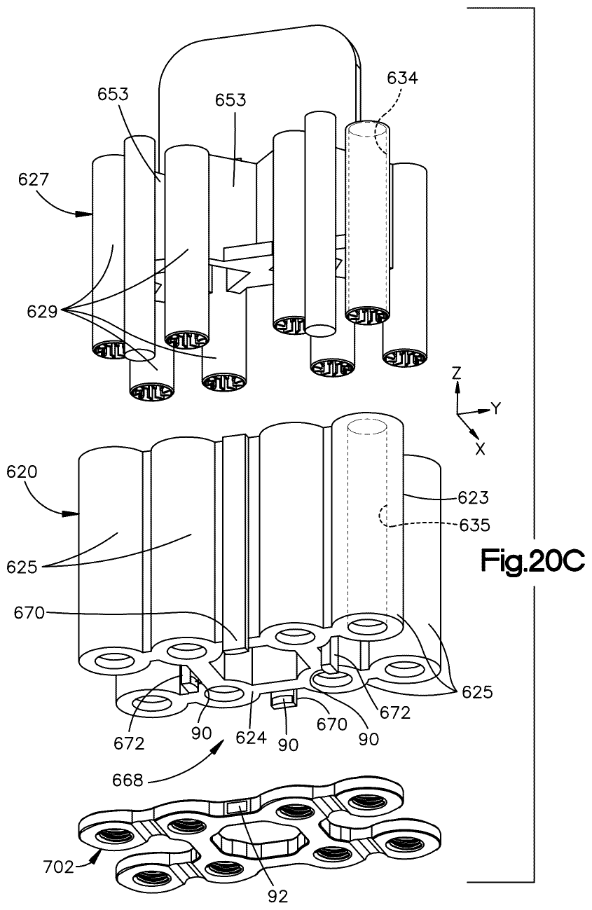

Referring now to FIGS. 20A-20C, an exemplary screw guide 604 is shown for coupling with an extended-area bone plate, such as an 8-hole H-plate 702, similar to the H-plate style bone plates 202, 202′ described above. The guide 604 of the present embodiment can be similar to the screw guide 504 described above with reference to FIGS. 19A-19C. As shown, the guide 604 can include eight (8) channels 634, which can be defined within barrel portions 629 of a cartridge 627 that is insertable within an outer housing 620, in similar fashion to guide 504. The outer housing 620 has opposed sidewalls 626, 628 that are spaced from each other along the second direction X and opposed sidewalls 630, 632 spaced from each other along the third direction Y. The interior space 639 of the outer housing 620 is defined between interior surfaces of the sidewalls 626, 628, 630, 632. The outer housing 620 defines tubular portions 625 having outer tube surfaces 633 and interior tube surfaces 635 opposite each other. The interior tube surfaces 635 define barrel receptacles 655 that are configured to receive the barrel portions 629 of the cartridge 627. The tubular portions 625 can also define slots 657 extending between the outer and interior tube surfaces 633, 635 for receiving bracket arms 653 of the cartridge 627 that interconnect the barrel portions 629 thereof.

The guide 604 has a coupling mechanism 668 that includes a first pair of flexible arms 670 extending downward from one pair of the opposed sidewalls (in the illustrated embodiment, from sidewalls 626, 628) and a second pair of flexible arms 672 extending downward from the other pair of the opposed sidewalls (e.g., 630, 632). The flexible arms 670, 672 have distal ends that include respective protrusions 90. In the illustrated embodiment, the protrusions of the first pair of flexible arms 670 face each other along the second direction X and protrusions of the second pair of flexible arms 672 face each other along the third direction Y. The protrusions 90 are configured to couple within associated recesses 92 in the exterior bridge side surfaces 58a of the bone plate 602. The spring arms 670 are flexible to facilitate easy coupling and decoupling with the bone plate 702.

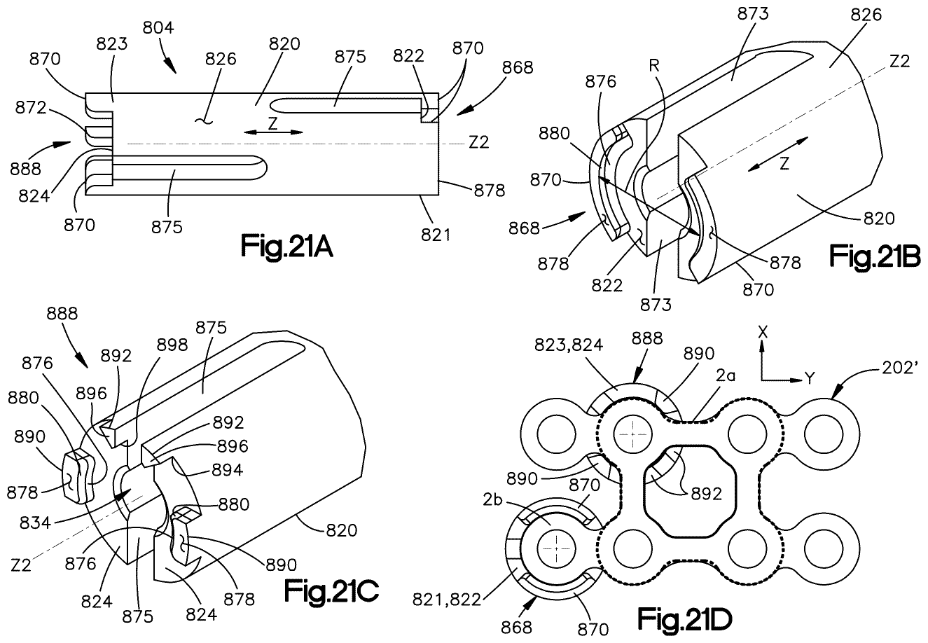

Referring now to FIGS. 21A-21D, an exemplary single-channel, dual-ended screw guide 804 is shown for coupling with individual holes 18 of a bone plate in different fashion depending on the location of the hole 18 in the bone plate. Although the bone plate 202′ shown in FIG. 21D is a double-H plate, as also shown in FIG. 16B, it should be appreciated that the dual-ended guide 804 can couple with various bone plate types and designs. The dual-ended guide 804 has a guide body 820 having opposed first and second ends 821, 823 having respective first and second coupling mechanisms 868, 888 that are each configured to couple with associated plate holes 18 depending on their location in the bone plate. The first end 821 and the second end 823 are spaced from each other along the first direction Z, which in this embodiment can be referred to as the “longitudinal” direction Z. The guide body 820 has an outer surface 826 extending longitudinally (i.e., along the longitudinal direction Z) between the first and second ends 821, 823. The guide body 820 defines a channel 834 that extends from a first end surface 822 at the first end 821 to a second end surface 824 at the second end 823 along a central channel axis Z2. The first and second coupling mechanisms 868, 888 have different configurations for coupling to holes 18 at different regions of the bone plate 202′. In the illustrated embodiment, the first coupling mechanism 868 is configured to couple with holes 18 located along an arm 2b of the bone plate 202′, and the second coupling mechanism 888 is configured to couple with holes 18 located along the hub portion 2a of the bone plate 202′. The guide body 820 can also include a screw retention mechanism, which can be similar to those described above.

The first coupling mechanism 868 includes a plurality of engagement members 870 that extend longitudinally outward from the first end surface 822. As shown, the plurality of engagement members 870 can include two (2) engagement members 870 located on opposite sides of the channel 834 and are opposite one another along a radial direction R that is perpendicular to the central channel axis Z2. The engagement members 870 each have an interior surface 876 configured to face and engage with an exterior side surface 58 of the bone plate. The engagement members 870 also have end surfaces 878 and chamfer surfaces 880 that extend from the interior surfaces 876 to the end surfaces 878. As shown, the interior surfaces 876 each extend circumferentially about the central channel axis Z2 and are configured to clamp against exterior node side surfaces 58b along an arm portion 2b of the bone plate 202′. The first coupling mechanism 868 also includes a first spring relief slot 873 defined in the guide body 820. The first spring relief slot 873 extends longitudinally from the first end surface 822 toward the second end 823 and extends from opposite sides of the outer surface 826 of the guide body 820, thereby bisecting a longitudinal portion of the guide body 820 extending to the first end 821. It should be appreciated that the first spring relief slot 873 can also act as a visualization window into the channel 834.

The second coupling mechanism 888 includes a second plurality of engagement members 890 and a third plurality of engagement members 892 each extending longitudinally outward from the second end surface 824. As shown, the second plurality of engagement members 890 can include two (2) engagement members 890 located on opposite sides of the channel 834 and are opposite one another along a respective radial direction R. The third plurality of engagement members 892 can include two (2) engagement members 892 located on opposite sides of the channel 834 from each other. The third plurality of engagement members 892 can be collectively spaced about 90-degrees intermediate the second plurality of engagement members 890 about the central channel axis Z2. The second plurality of engagement members 890 have respective interior surface 876 configured to engage respective exterior side surfaces 58 of the bone plate. The second plurality of engagement members 890 have respective end surfaces 878 and respective chamfer surfaces 880 that extend from the interior surfaces 876 to the end surfaces 880. The third plurality of engagement members 892 have respective interior surfaces 894, end surfaces 896, and chamfer surfaces 898 extending therebetween. As shown, the interior surfaces 876 of the second plurality of engagement members 890 are configured to clamp against exterior and interior node side surfaces 58b, 60b along the hub portion 2a of the bone plate 202′. The interior surfaces 894 of the third plurality of engagement members 892 are configured to primarily support against exterior and interior relief side surfaces 58c, 60c along the hub portion 2a. The second coupling mechanism 868 also includes a second spring relief slot 875 that is defined in the guide body 820 at the second end 823 and is configured similar to the first spring relief slot 873. Accordingly, the second spring relief slot 875 can also act as a visualization window into the channel 834.

The first and second coupling mechanisms 868, 888 couple with the bone plate in generally similar fashion to each other. Guide-to-plate coupling involves advancing the first end 821 or second end 823, respectively, toward the target plate hole 18, preferably with the central channel axis Z2 substantially coaxial with the respective central hole axis Z1. The surgeon orients or otherwise indexes the guide body 820 (i.e., rotates the guide body 820 about the central channel axis Z2) as needed so that the engagement members 870, 890, 892 of the respective coupling mechanism 868, 888 align with the associated side surfaces 58, 60 of the bone plate 202′. As the respective coupling mechanism 868, 888 advances into engagement with the bone plate 202′ at a proper orientation, the chamfer surfaces 880 of the respective engagement members 870, 890, 892 engage the outer plate surface 14 and/or the upper chamfer surfaces 61, 63 of the bone plate 202′, which have a centering effect that facilities coaxial alignment with the target hole 18. Additionally, engagement between the chamfer surfaces 880 of the first and second coupling mechanisms 868, 888, respectively and the plate chamfer surfaces 61, 63 pushes the engagement members 870, 890 away from each other, as facilitated by the first or second spring relief slot 873, 875 as the first or second end 821, 823 further advances against the bone plate 202′. This, in turn, imparts the interior surfaces 876 with a return clamping force, which is applied against the exterior side surfaces 58 of the bone plate 202′ once the interior surfaces 876 descend into engagement with the exterior side surfaces 58.

It should be appreciated that the various bone plates described above can include additional or alternative features, such as bend-enhancing features (e.g., areas of reduced cross-sectional dimension), to facilitate contouring or otherwise shaping the bone plates to the underlying bone.

It should also be appreciated that in additional embodiments, the guides, bone plates, and bone fasteners (e.g., bone screws) can be provided in a kit that includes a plurality of guides that are interchangeable with various bone plates having the hub structure 2a described above yet also having different plate portions (e.g., arms 2b) extending from the hub structure 2a.

Although the foregoing disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

1. A bone plating assembly for bone fixation, comprising:
a bone plate having a plate body that has an outer surface and a bone-facing surface opposite the outer surface, the plate body defining at least one plate hole that extends from the outer surface to the bone facing surface along a central hole axis; and
a guide body having a proximal end and a distal end spaced from each other along a direction, the distal end having a coupling mechanism for coupling to the bone plate at an orientation of the guide body, the guide body defining at least one channel that extends along a central channel axis and is configured to receive at least one respective bone fastener therein in a pre-loaded position,
wherein the guide body is configured such that the central channel axis is substantially coaxial with the central hole axis when the guide body is connected to the bone plate at the orientation, and
wherein the guide body includes a retention mechanism within the at least one channel, the retention mechanism configured to apply a retention force to the at least one respective bone fastener for retaining the at least one respective bone fastener within the at least one channel in the pre-loaded position.

2. The bone plating assembly of claim 1, wherein the retention mechanism comprises a flexible tab defined by a cutout portion of the guide body, the flexible tab being biased inward toward the central hole axis such that an interior surface of the flexible tab is configured to contact a head of the at least one respective bone fastener to thereby apply the retention force thereto in the pre-loaded position.

3. The bone plating assembly of claim 1, wherein the retention mechanism comprises interior threading defined on an interior surface of the at least one channel, the interior threading configured to temporarily engage exterior threads defined on a head of the at least one bone fastener in the pre-loaded position.

4. The bone plating assembly of claim 1, wherein:
the plate body defines a plurality of side surfaces extending between the outer surface and the bone-facing surface; and
the coupling mechanism comprising at least two engagement members that are configured to extend alongside and couple with at least two of the plurality of side surfaces of the plate body with sufficient coupling force to couple the plate body with the guide body at least against the force of gravity.

5. The bone plating assembly of claim 4, wherein the at least two engagement members are configured to extend alongside and couple with the at least two of the plurality of side surfaces of the plate body, wherein the at least two of the plurality of side surfaces are opposed exterior side surfaces of the plate body.

6. The bone plating assembly of claim 5, wherein the guide body comprises a spring member configured to bias the at least two engagement members toward each other and toward the respective opposed exterior side surfaces of the plate body.

7. The bone plating assembly of claim 6, wherein:
the plate body defines an aperture spaced from the at least one plate hole, the aperture extending from the outer surface to the bone-facing surface; and
the coupling mechanism includes two inner engagement members that are configured to extend alongside and couple with another two of the plurality of side surfaces of the plate body, the another two of the plurality of side surfaces located within the aperture.

8. The bone plating assembly of claim 7, wherein:
the at least one plate hole comprises four plate holes extending from the outer surface to the bone facing surface along respective central hole axes, the four plate holes arranged in a square pattern, the plate body including four nodes in which the four plate holes are respectively defined,
the four nodes are interconnected by four bridge members,
the aperture is centrally located between the four plate holes, and
the at least one channel comprises four channels extending from the proximal end to the distal end of the guide body along respective central channel axes, each of the channels extending coaxially with a respective one of the plate holes when the guide body is at the orientation.

9. The bone plating assembly of claim 4, wherein:
the at least two of the plurality of side surfaces of the plate body define respective recess therein; and
the at least two engagement members include respective protrusions configured to extend within the respective recesses.

10. The bone plating assembly of claim 1, further comprising a handle that is repeatedly attachable to and detachable from the guide body, the handle extending from a free end to an attachment end, the attachment end having a mounting structure configured to attach to and detach from a complimentary mounting formation defined along a side of the guide body, the side extending between the proximal and distal ends along the direction.

11. The plating assembly of claim 1, wherein:
the at least one channel of the guide body is a single channel configured to receive a single bone fastener therein at a given time,
the distal end of the guide body comprises four engagement members that are arranged in a rectangular pattern and are configured to extend alongside respective side surface portions of the plate body in a manner maintaining the angular orientation of the guide body within a limited angular range of rotation relative to the bone plate about the central hole axis.

12. The plating assembly of claim 1, wherein:
the at least one channel of the guide body is a single channel configured to receive a single bone fastener therein at a given time,
the distal end of the guide body having a first coupling mechanism configured to contact the plate body for connection thereto at the orientation, the orientation being a first orientation,
the proximal end of the guide body having a second coupling mechanism configured to contact the plate body for connection thereto at a second orientation,
the first and second coupling mechanisms each comprise at least two engagement members that are configured to extend alongside and clamp against at least two opposed surface portions of the plate body, the at least two opposed surface portions extending between the outer and bone-facing surfaces of the plate body along the direction when the guide body is connected to the bone plate at the first and second orientations, and
the at least two engagement members of the first coupling mechanism are configured to press toward each other against at least two opposed surface portions that are exterior surface portions of the plate body remote from the at least one plate hole, and
the at least two engagement members of the second coupling mechanism are configured to press toward each other against at least two opposed surface portions that are exterior surface portions of the plate body remote from the at least one plate hole.

13. A screw guide for connecting to a bone plate having at least one fastener hole, comprising:
a body having a proximal end and a distal end spaced from each other along a direction, the distal end having a coupling mechanism for coupling the body to the bone plate at an orientation, the body defining at least one channel that extends along a central channel axis, the at least one channel configured to receive at least one respective bone fastener therein in a pre-loaded position,
wherein the coupling mechanism is configured such that the central channel axis substantially aligns with a central hole axis of the at least one fastener hole when the coupling mechanism is coupled to the bone plate at the orientation, and
wherein the body has a retention mechanism at least partially located within the at least one channel, the retention mechanism configured to apply a retention force to the at least one respective bone fastener for retaining the at least one respective bone fastener within the at least one channel in the pre-loaded position.

14. The screw guide of claim 13, wherein the retention mechanism comprises a flexible tab defined by a cutout portion of the body, the flexible tab being biased inward toward the central hole axis such that an interior surface of the flexible tab is configured to contact a head of the at least one respective bone fastener to thereby apply the retention force thereto in the pre-loaded position.

15. The screw guide of claim 13, wherein the retention mechanism comprises interior threading defined on an interior surface of the at least one channel, the interior threading configured to temporarily engage exterior threads defined on a head of the at least one bone fastener in the pre-loaded position.

16. The screw guide of claim 13, wherein the coupling mechanism comprising a pair of engagement members that are configured to extend alongside and clamp against at least two opposed side surfaces of the bone plate.

17. The screw guide of claim 16, wherein the pair of engagement members each comprise a bottom surface, an interior surface that extends along the direction between a bottom surface of the body and the bottom surface of the respective engagement member, and a chamfer surface that extends from the interior surface to the bottom surface of the engagement member, wherein the chamfer surfaces of the pair of engagement members are configured to push the pair of engagement members away from each other as the chamfer surfaces engage the bone plate.

18. The bone plating assembly of claim 16, wherein the body comprises a spring member configured to bias the pair of engagement members toward each other for clamping against the respective opposed exterior side surfaces of the plate body.

19. The screw guide of claim 18, wherein the coupling mechanism includes two additional engagement members that are positioned between the pair of engagement members, the two additional engagement members being configured to extend alongside and couple with another two side surfaces of the plate body for centering the body at the orientation relative to the bone plate.

20. The screw guide of claim 19, wherein the at least one channel comprises four channels extending from the proximal end to the distal end of the body along respective central channel axes, the four channels being arranged in a square pattern, wherein the retention mechanism comprises four compliant members each defined by the body and each extending within a respective one of the four channels.
